(19) United States
(12) Patent Application Publication
(10) Pub. No.: US 2023/0243843 A1
(43) Pub. Date: Aug. 3, 2023

(54) SEQUENCE VARIANCE ANALYSIS BY PROTEOMINER

(71) Applicant: Regeneron Pharmaceuticals, Inc., Tarrytown, NY (US)

(72) Inventors: Sisi Zhang, White Plains, NY (US); Bo Zhao, White Plains, NY (US); Hui Xiao, Ridgewood, NJ (US); Ning Li, New Canaan, CT (US)

(21) Appl. No.: 18/093,936

(22) Filed: Jan. 6, 2023

Related U.S. Application Data
(60) Provisional application No. 63/297,822, filed on Jan. 10, 2022.
(60) Provisional application No. 63/426,199, filed on Nov. 17, 2022.
(60) Provisional application No. 63/433,106, filed on Dec. 16, 2022.

(51) Int. Cl. G01N 33/68; G01N 30/72

(52) U.S. Cl. CPC G01N 33/6848; G01N 30/7233; G01N 2030/8831

(57) ABSTRACT

The present invention provides methods and systems to identify host cell protein (HCP) impurities in a sample containing high-abundance proteins. The HCP impurities can be enriched using interacting peptide ligands which have been attached to solid support. The HCP impurities can be eluted from the solid support. The isolated HCP impurities can be subjected to limited digestion to generate components of the isolated HCP impurities which can subsequently be identified using a mass spectrometer. The present invention also provides methods and systems to identify sequence variant (SV) peptides or proteins in a sample containing high-abundance proteins. The SV peptides or proteins can be enriched using interacting peptide ligands which have been attached to solid support. The SV peptides or proteins can be eluted from the solid support. The isolated SV peptides or proteins can be subjected to full or limited digestion to generate components of the isolated SV peptides or proteins which can subsequently be identified using a mass spectrometer.

### CROSS-REFERENCE TO RELATED APPLICATIONS

This and claims priority to and the benefit of Provisional Patent Application No. 63/297,822, filed on Jan. 10, 2022, U.S. Provisional Patent Application No. 63/426,199, filed on Nov. 17, 2022, and U.S. Provisional Patent Application No. 63/433,106, filed on Dec. 16, 2022, the content of which is incorporated herein by reference in its entirety.

### SEQUENCE LISTING

The instant application contains a Sequence Listing which has been submitted electronically in XML format and is hereby incorporated by reference in its entirety. Said XML copy, created on Mar. 30, 2023, is named 070816-02962_11105US01_SL.xml and is 26,612 bytes in size.

### FIELD

The present invention generally pertains to methods for identifying and quantitating low-abundance host cell proteins (HCP) to monitor and control impurities in biopharmaceutical products. The present invention also relates to methods for enriching, identifying, and quantitating amino acid sequence variant (SV) proteins in biopharmaceutical products.

### BACKGROUND

Recombinant DNA technology has been used widely for producing biopharmaceutical products in host cells. Biopharmaceutical products must meet very high standards of purity. Thus, it can be important to monitor any impurities in such biopharmaceutical products at different stages of drug development, production, storage and handling. Residual impurities should be at an acceptable low level prior to conducting clinical studies. Residual impurities are also a concern for biopharmaceutical products intended for end-users. For example, host cell proteins (HCPs) can be present in protein-based biopharmaceuticals which are developed using cell-based systems. The presence of HCPs in drug products needs to be monitored and can be unacceptable above a certain threshold, depending on the product and the particular HCP. Sometimes, even trace amounts of HCPs can cause an immunogenic response.

Immuno-assays have been used to monitor HCP removal using polyclonal anti-HCP antibodies. Immuno-assays can provide semi-quantitation of total HCP levels in high throughput, but they may not be effective in quantitating individual HCPs rapidly. Liquid chromatography-mass spectrometry (LC-MS) has recently emerged for monitoring HCP removal. However, the enormous dynamic concentration ranges of HCPs in the presence of a high concentration of purified antibodies can be a challenge for developing LC-MS methods to monitor the removal of HCPs. In particular, quantifying individual HCPs at extremely low levels (<1 ppm) is challenging.

It will be appreciated that a need exists for methods and systems to identify and quantitate HCPs to monitor and control the residual HCPs in a drug substance or other product to mitigate safety risks.

Sequence variants (SVs) resulting from unintended amino acid substitutions in recombinant therapeutic proteins have increasingly gained attention from both regulatory bodies and the biopharmaceutical industry, given the potential impact on efficacy and safety. With well-optimized production systems, such sequence variants usually exist at very low levels in final products due to the high fidelity of DNA replication and protein biosynthesis processes in mammalian expression systems such as Chinese hamster ovary (CHO) cell lines. However, SV levels can be significantly elevated in cases where the selected production cell line has unexpected DNA mutations or the manufacturing process is not fully optimized, for example, if depletion of certain amino acids occurs in the cell culture media in bioreactors. Therefore, it is important to design and implement an effective monitoring and control strategy to prevent or minimize the possible risks of SVs during the early stage of product and process development. However, there is no well-established guidance from the regulatory bodies or consensus across the industry to assess and manage SV risks.

The biopharmaceutical industry currently targets a general control limit of 0.1% sequence variation of individual amino acids in therapeutic monoclonal antibodies (mAbs), which appears to be the upper limit of natural sequence variation of individual amino acids. However, there is not a sensitive, accurate, and precise method for detecting SV proteins. For example, three independent laboratories digested NIST standard mAbs, purified the NIST mAb tryptic peptides using regular flow charge surface hybrid (CSH) LC columns, and detected SV NIST mAb tryptic peptides using mass spectrometry (Zhang, et al. 2020). The three laboratories each identified 21-23 sequence variations in the NIST monoclonal antibody (mAb) at a rate between 0.01% and 0.1%, but the laboratories were only in agreement in respect to 12 sequence variations. A need exists for more reproducible and reliable methods of detecting the full array of SV proteins within biopharmaceutical therapies, especially at a 0.1% sequence variation for individual amino acids as an upper limit of impurity.

It will be appreciated that a need exists for methods and systems to identify and quantitate amino acid sequence variations within biotherapeutics to ensure drug product safety, consistency, and efficacy.

### SUMMARY

The identification of HCP impurities in biopharmaceutical products is challenging due to the broad dynamic range of protein concentrations in samples with very high complexity. In particular, the presence of at least one high-abundance protein or peptide in a sample, such as a therapeutic protein, creates technical obstacles to the detection, identification and quantification of very low-abundance proteins in a sample. The present application provides methods to identify HCP impurities in a sample containing high-abundance proteins, including an enrichment method to fulfill the need of enriching low abundance HCPs in therapeutic drug products.

This disclosure provides methods of identifying and/or quantifying HCP impurities in a sample. In some exemplary embodiments, the method comprises: (a) contacting a sample including at least one high-abundance peptide or protein and at least one HCP impurity to a solid support, wherein said solid support is attached to interacting peptide ligands capable of interacting with said at least one HCP impurity; (b) washing said solid support to provide an eluate comprising at least one enriched HCP impurity; (c) subjecting said eluate to an enzymatic digestion condition to generate at least one component of said at least one enriched HCP impurity, wherein said enzymatic digestion condition does not fully digest all proteins in said eluate; (d) identifying said at least one component of said at least one enriched HCP impurity using a mass spectrometer; and (e) using the identification of said at least one component to identify said at least one enriched HCP impurity.

In one aspect, the washing step includes a surfactant, wherein said surfactant is a phase transfer surfactant, an ionic surfactant, an anionic surfactant, a cationic surfactant, or combinations thereof. In a specific aspect, the surfactant is sodium deoxycholate, sodium lauryl sulfate, sodium dodecylbenzene sulphonate, or combinations thereof. In another aspect, a concentration of the surfactant is about 12 mM. In a specific aspect, the surfactant comprises about 12 mM sodium deoxycholate and about 12 mM sodium lauryl sulfate.

In one aspect, a concentration of the at least one high-abundance peptide or protein is at least about 1000 times, about 10,000 times, about 100,000 times or about 1,000,000 times higher than a concentration of said at least one HCP impurity. In another aspect, the interacting peptide ligands are a library of combinatorial hexapeptide ligands. In yet another aspect, the at least one high-abundance peptide or protein is an antibody, a bispecific antibody, an antibody fragment, a Fab region of an antibody, an antibody-drug conjugate, a fusion protein, a recombinant protein, a protein pharmaceutical product, a biopharmaceutical product, or a drug.

In one aspect, an enzyme of the enzymatic digestion condition is trypsin. In a specific aspect, the enzymatic digestion condition includes trypsin at an enzyme to substrate ratio of less than about 1:200. In another specific aspect, the enzymatic digestion condition includes trypsin at an enzyme to substrate ratio of about 1:400, about 1:1000, about 1:2500, or about 1:10000. In another aspect, the at least one enriched HCP impurity is not subjected to denaturation prior to being subjected to said enzymatic digestion condition.

In one aspect, the mass spectrometer is an electrospray ionization mass spectrometer, nano-electrospray ionization mass spectrometer, or a triple quadrupole mass spectrometer, wherein the mass spectrometer is coupled to a liquid chromatography system. In another aspect, the mass spectrometer is capable of performing LC-MS (liquid chromatography-mass spectrometry) or a LC-MRM-MS (liquid chromatography-multiple reaction monitoring-mass spectrometry) analyses.

In one aspect, the method further comprises quantifying the at least one enriched HCP impurity using said mass spectrometer, wherein a detection limit of the at least one enriched HCP impurity is about 0.003-0.006 ppm.

Existing similar detection methods cannot detect the same array of amino acid sequence variations within the same NIST mAb standard (Zhang, et al. 2020). A potential explanation may be that aberrant amino acid substitutions can occur at any amino acid within the sequence of a protein, creating a diverse array of sequence variations that evade reliable identification. Therefore, quantitating risks associated with SV proteins generally, as well as specific SV proteins or subsets of SV proteins, is not possible using existing methods. The present application presents a method that can identify approximately four times as many sequence variations within the same NIST mAb standard as multiple previous studies. Furthermore, the methods of the present disclosure are particularly suited for reproducibly identifying amino acid sequence variations that are likely to affect three-dimensional protein structure. Specifically, the ProteoMiner™ SV identification method of the present disclosure most effectively enriches SV proteins in which an amino acid with a physical characteristic, like the negatively charged polar side chain of glutamic acid, replaces an amino acid with a different physical characteristic, like the nonpolar hydrophobic side chain of valine.

This disclosure provides methods for identifying SV peptides or proteins in a sample, wherein at least one amino acid of a SV peptide or protein unintentionally differs from a wild-type peptide or protein. In some embodiments, the method comprises: (a) contacting a sample including at least one more-abundant wild-type peptide or protein and at least one SV peptide or protein to a solid support, wherein the solid support is attached to interacting peptide ligands capable of interacting with the at least one SV peptide or protein; (b) washing the solid support to provide a first eluate comprising at least one enriched SV peptide or protein; (c) subjecting the first eluate to an enzymatic digestion condition to generate at least one component of the at least one enriched SV peptide or protein; (d) subjecting the first eluate with the at least one component of the at least one enriched SV peptide or protein to a liquid chromatography system to produce a second eluate with the at least one component of the at least one enriched SV peptide or protein; (e) subjecting the second eluate with the at least one component of the at least one enriched SV peptide or protein to mass spectrometry; (f) identifying the at least one component of the at least one enriched SV peptide or protein using a mass spectrometer; and (g) using the identification of the at least one component of the at least one enriched SV peptide or protein to identify the at least one enriched SV peptide or protein in the sample.

In one aspect, the enzymatic digestion condition is a direct digestion.

In one aspect, the liquid chromatography system comprises a nanoscale liquid chromatography (nanoLC) column or a regular flow CSH column.

In one aspect, the enzymatic digestion condition does not fully digest all proteins in the first eluate.

In one aspect, the solid support is washed using a surfactant, wherein the surfactant is a phase transfer surfactant, an ionic surfactant, an anionic surfactant, a cationic surfactant, or combinations thereof.

In one aspect, the surfactant is sodium deoxycholate, sodium lauryl sulfate, sodium dodecylbenzene sulphonate, or combinations thereof.

In one aspect, a concentration of the surfactant is about 12 mM.

In one aspect, the surfactant comprises about 12 mM sodium deoxycholate and about 12 mM sodium lauryl sulfate.

In one aspect, a concentration of the at least one more-abundant wild-type peptide or protein is at least about 1000 times, about 10,000 times, about 100,000 times or about 1,000,000 times higher than a concentration of the at least one SV peptide or protein.

In one aspect, the interacting peptide ligands are a library of combinatorial hexapeptide ligands.

In one aspect, the at least one more-abundant wild-type peptide or protein and the at least one SV peptide or protein are an antibody, a bispecific antibody, an antibody fragment, a Fab region of an antibody, an antibody-drug conjugate, a fusion protein, a recombinant protein, a protein pharmaceutical product, a biopharmaceutical product, or a drug.

In one aspect, an enzyme of the enzymatic digestion condition is trypsin.

In one aspect, the enzymatic digestion condition includes trypsin at an enzyme to substrate ratio of less than about 1:200.

In one aspect, the enzymatic digestion condition includes trypsin at an enzyme to substrate ratio of about 1:400, about 1:1000, about 1:2500, or about 1:10000.

In one aspect, the at least one enriched SV peptide or protein is not subjected to denaturation prior to being subjected to the enzymatic digestion condition.

In one aspect, the mass spectrometer is an electrospray ionization mass spectrometer, nano-electrospray ionization mass spectrometer, or a triple quadrupole mass spectrometer, wherein the mass spectrometer is coupled to the liquid chromatography system.

In one aspect, the mass spectrometer is capable of performing LC-MS (liquid chromatography-mass spectrometry) or LC-MRM-MS (liquid chromatography-multiple reaction monitoring-mass spectrometry) analyses.

In one aspect, the method further comprises quantifying the at least one enriched SV peptide or protein using the mass spectrometer, wherein a detection limit of the at least one enriched SV peptide or protein is about 0.003-0.006 ppm.

The present disclosure provides methods for identifying host cell protein (HCP) impurities in a sample. In some embodiments, the method comprises: (a) contacting a sample including at least one high-abundance peptide or protein and at least one HCP impurity to a solid support, wherein said solid support is attached to interacting peptide ligands capable of interacting with said at least one HCP impurity; (b) washing said solid support to provide an eluate comprising at least one enriched HCP impurity; (c) subjecting said eluate to an enzymatic digestion condition to generate at least one component of said at least one enriched HCP impurity, wherein said enzymatic digestion condition does not fully digest all proteins in said eluate; (d) identifying said at least one component of said at least one enriched HCP impurity using parallel reaction monitoring-mass spectrometry; and (e) using the identification of said at least one component to identify said at least one enriched HCP impurity.

In one aspect, the solid support is washed using a surfactant, wherein the surfactant is a phase transfer surfactant, an ionic surfactant, an anionic surfactant, a cationic surfactant, or combinations thereof.

In another aspect, the surfactant is sodium deoxycholate, sodium lauryl sulfate, sodium dodecylbenzene sulphonate, or combinations thereof.

In one aspect, a concentration of the surfactant is about 12 mM.

In yet another aspect, the surfactant comprises about 12 mM sodium deoxycholate and about 12 mM sodium lauryl sulfate.

In one aspect, a concentration of the at least one high-abundance peptide or protein is at least about 1,000 times, about 10,000 times, about 100,000 times, about 1,000,000 times, about 10,000,000 times, about 100,000,000 times or about 1,000,000,000 times higher than a concentration of said at least one HCP impurity.

In one aspect, the interacting peptide ligands are a library of combinatorial hexapeptide ligands.

In one aspect, the at least one high-abundance peptide or protein is an antibody, a bispecific antibody, an antibody fragment, a Fab region of an antibody, an antibody-drug conjugate, a fusion protein, a recombinant protein, a protein pharmaceutical product, or a drug.

In one aspect, an enzyme of said enzymatic digestion condition is trypsin.

In another aspect, the enzymatic digestion condition includes trypsin at an enzyme to substrate ratio of less than about 1:200.

In yet another aspect, the enzymatic digestion condition includes trypsin at an enzyme to substrate ratio of about 1:400, about 1:1000, about 1:2500, or about 1:10000.

In one aspect, the at least one enriched HCP impurity is not subjected to denaturation prior to being subjected to said enzymatic digestion condition.

In one aspect, the mass spectrometer is an electrospray ionization mass spectrometer, nano-electrospray ionization mass spectrometer, or a triple quadrupole mass spectrometer, wherein the mass spectrometer is coupled to a liquid chromatography system.

In one aspect, the sample includes an internal standard.

In another aspect, the internal standard is labeled with a heavy isotope.

In yet another aspect, the internal standard is hPLBD2.

These, and other, aspects of the present invention will be better appreciated and understood when considered in conjunction with the following description and the accompanying drawings. The following description, while indicating various embodiments and numerous specific details thereof, is given by way of illustration and not of limitation. Many substitutions, modifications, additions, or rearrangements may be made within the scope of the present invention.

### BRIEF DESCRIPTION OF THE DRAWINGS

FIG. 1 illustrates a workflow of the method of the present invention, according to an exemplary embodiment.

FIG. 2 shows the number of HCPs and UPS2 proteins identified by alternative ProteoMiner™ limited digestion methods, according to an exemplary embodiment.

FIG. 3 shows the number of HCPs and UPS2 proteins identified by the ProteoMiner™ limited digestion method with a range of trypsin to substrate ratios, according to an exemplary embodiment.

FIG. 4 shows the number of HCPs and UPS2 proteins identified by the ProteoMiner™ limited digestion method with SDC/SLS presented at a range of 2.4 mM to 12 mM, according to an exemplary embodiment.

FIG. 5A shows the UPS2 proteins identified by the previously described ProteoMiner™ method, optimized limited digestion method and the ProteoMiner™ limited digestion method of the present invention, according to an exemplary embodiment. FIG. 5B shows additional UPS2 proteins identified by the previously described ProteoMiner™ method, optimized limited digestion method and the ProteoMiner™ limited digestion method of the present invention, according to an exemplary embodiment.

FIG. 6 shows the number of HCPs identified by the optimized ProteoMiner™ limited digestion method of the present invention compared to conventional methods, according to an exemplary embodiment.

FIG. 7 shows the number of UPS2 proteins identified by the optimized ProteoMiner™ limited digestion method of the present invention compared to conventional methods, according to an exemplary embodiment.

FIG. 8 shows the number of NIST monoclonal antibody (mAb) HCPs identified by the optimized ProteoMiner™ limited digestion method of the present invention compared to previously published methods, according to an exemplary embodiment.

FIG. 9 illustrates a sample preparation workflow for the ProteoMiner™ and ultrasensitive quantification method for enhanced detection of host cell proteins, according to an exemplary embodiment.

FIG. 10A shows a comparison of targeted quantification (PRM) of GLGDVDQLVK (SEQ ID NO: 1) from LPL in mAb-1 and mAb-1 with the corresponding recombinant standard spiked in at the lower limit of quantitation (LLOQ) level, according to an exemplary embodiment.

FIG. 10B shows a comparison of targeted quantification (PRM) of EFSHITFLTIK (SEQ ID NO: 2) from carboxypeptidase in mAb-1 and mAb-1 with the corresponding recombinant standard spiked in at the lower limit of quantitation (LLOQ) level, according to an exemplary embodiment.

FIG. 10C shows a comparison of targeted quantification (PRM) of VNVYTSHSPAGTSVQNLR (SEQ ID NO: 3) from LAL in mAb-1 and mAb-1 with the corresponding recombinant standard spiked in at the lower limit of quantitation (LLOQ) level, according to an exemplary embodiment.

FIG. 10D shows a comparison of targeted quantification (PRM) of VSSLPSVTLK (SEQ ID NO: 4) from cathepsin D in mAb-1 and mAb-1 with the corresponding recombinant standard spiked in at the lower limit of quantitation (LLOQ) level, according to an exemplary embodiment.

FIG. 10E shows a comparison of targeted quantification (PRM) of GVNYASITR (SEQ ID NO: 5) from cathepsin Z in mAb-1 and mAb-1 with the corresponding recombinant standard spiked in at the lower limit of quantitation (LLOQ) level, according to an exemplary embodiment.

FIG. 11A shows the standard curve for eight peptides, according to an exemplary embodiment. Peak area ratio (PAR) was calculated according to the peak area chosen for each HCP divided by the peak area of the peptide from hPLBD2 from PRM analysis. HCPs were spiked into mAb-1. The list of peptides from HCPs and hPLBD2 is shown in Table 4.

FIG. 11B shows the standard curve of peak area ratio (PAR) for two peptides chosen for LAL and human PPT-1, divided by the peptide from hPLBD2 from PRM analysis, according to an exemplary embodiment. LAL and human PPT-1 were spiked into mAb-2.

FIG. 12A shows the PS80 degradation profile observed in mAb-3 (1.28 ppm LAL and 0.2 ppm LPL present) under normal storage conditions (4° C.-8° C.) for up to 6 months, on the basis of LC-CAD measurements, according to an exemplary embodiment.

FIG. 12B shows the increased concentration of oleic acid observed in mAb-3 (1.28 ppm LAL and 0.2 ppm LPL present) under normal storage conditions (4° C.-8° C.), on the basis of free fatty acid measurements, according to an exemplary embodiment.

FIG. 12C shows correlations between oleic acid increase per day under stress conditions (37° C.) and lipase concentrations (LAL and LPL) in mAb-3 to mAb-10, according to an exemplary embodiment.

FIG. 13 shows correlations between oleic acid increase per day under stress conditions (37° C.) and measured CES concentrations in mAb-12 to mAb-17, according to an exemplary embodiment. CES concentrations in mAb-15 to mAb-17 were quantified by comparison of the relative abundance of CES to mAb-13 and mAb-14.

FIG. 14 shows correlation between remaining PS80% under storage conditions (4-8° C.) for mAb-18, according to an exemplary embodiment.

FIG. 15A shows MY truncation under stress conditions (45° C.) observed for DS-1, DS-2 and DS-3 up to 6 months, according to an exemplary embodiment.

FIG. 15B shows concentrations of cathepsin D in DS-1, DS-2 and DS-3, according to an exemplary embodiment.

FIG. 16 shows that potential mechanisms for producing SV proteins may occur during replication, transcription, translation, or a combination thereof, according to an exemplary embodiment.

FIG. 17 illustrates a workflow of the enhanced SV protein detection method of the present invention, according to an exemplary embodiment.

FIG. 18A shows a table of amino acid substitutions identified in SV NIST mAbs using the ProteoMiner™ SV identification method of the present disclosure with nanoLC columns or direct digestions with nanoLC columns, according to an exemplary embodiment.

FIG. 18B shows a table of amino acid substitutions identified in SV NIST mAbs using the ProteoMiner™ SV identification method of the present disclosure with nanoLC columns or direct digestions with nanoLC columns, according to an exemplary embodiment.

FIG. 18C shows a table of amino acid substitutions identified in SV NIST mAbs using the ProteoMiner™ SV identification method of the present disclosure with nanoLC columns or direct digestions with nanoLC columns, according to an exemplary embodiment.

FIG. 19A shows a table of the enriched SV NIST mAb peptides identified using the ProteoMiner™ SV enrichment method of the present disclosure, according to an exemplary embodiment. Figure discloses SEQ ID NOS 16-21, and 17, respectively, in order of appearance.

FIG. 19B shows a view of the NIST mAb amino acid sequence variations enriched using the ProteoMiner™ SV identification method of the present disclosure within the three-dimensional protein structure of an SV NIST mAb, according to an exemplary embodiment.

FIG. 19C shows an alternative view of the NIST mAb amino acid sequence variations enriched using the ProteoMiner™ SV identification method of the present disclosure within the three-dimensional protein structure of an SV NIST mAb, according to an exemplary embodiment.

FIG. 19D shows another alternative view of the SV NIST mAb amino acid sequence variations identified using the ProteoMiner™ SV identification method of the present disclosure within the three-dimensional protein structure of an SV NIST mAb, according to an exemplary embodiment.

FIG. 20A shows the differing properties of histidine (e.g., positively charged side chain), asparagine (e.g., polar uncharged side chain), and aspartic acid (e.g., negatively charged side chain) responsible for histidine to asparagine or aspartic acid sequence variations affecting the protein structures of SV mAbs enriched by the ProteoMiner™ SV identification method of the present disclosure, according to an exemplary embodiment.

FIG. 20B shows the codon sequence variations that can cause histidine to asparagine or aspartic acid sequence variations in SV mAbs enriched by the ProteoMiner™ SV identification method of the present disclosure, according to an exemplary embodiment.

FIG. 20C shows the NIST mAb histidine to asparagine or aspartic acid sequence variations identified using eluates from NIST mAb direct digests subjected to regular flow CSH LC or nanoLC columns or ProteoMiner™ enriched NIST mAb digests subjected to nanoLC columns, according to an exemplary embodiment.

FIG. 20D shows the MS2 mass spectrum of tryptic peptide product ions detected in an eluate from an NIST mAb direct digest subjected to a regular flow CSH LC column (bottom), and the MS2 mass spectrum of histidine to asparagine SV tryptic peptide product ions detected in an eluate from a ProteoMiner™ enriched NIST mAb digest subjected to a nanoLC column (top), according to an exemplary embodiment. Figure discloses SEQ ID NOS 22-23, respectively, in order of appearance.

FIG. 20E shows the MS2 mass spectrum of tryptic peptide product ions detected in an eluate from an NIST mAb direct digest subjected to a regular flow CSH LC column (bottom), and the MS2 mass spectrum of histidine to aspartic acid SV tryptic peptide product ions detected in an eluate from a ProteoMiner™ enriched NIST mAb digest subjected to a nanoLC column (top), according to an exemplary embodiment. Figure discloses SEQ ID NOS 24 and 23, respectively, in order of appearance.

FIG. 20F shows the CHO IgG1 mAb histidine to asparagine or aspartic acid sequence variations identified using eluates from a CHO IgG1 direct digest subjected to regular flow CSH LC columns or ProteoMiner™ enriched CHO IgG1 mAb digests subjected to nanoLC columns, according to an exemplary embodiment.

FIG. 21A shows the similar properties of serine (e.g., polar uncharged side chain) and asparagine (e.g., polar uncharged side chain) that prevent serine to asparagine sequence variations from affecting the protein structures of SV mAbs not enriched by the enhanced ProteoMiner™ SV identification method of the present disclosure, according to an exemplary embodiment.

FIG. 21B shows the shows the NIST mAb serine to asparagine sequence variations identified using eluates from NIST mAb direct digests subjected to regular flow CSH LC or nanoLC columns or digested ProteoMiner™ NIST mAb eluates subjected to nanoLC columns, according to an exemplary embodiment.

FIG. 22A shows the number of NIST mAb amino acid sequence variations identified (SVA >0.01%) using eluates from NIST mAb direct digests subjected to regular flow CSH LC or nanoLC columns or digested ProteoMiner™ NIST mAb eluates subjected to nanoLC columns, according to an exemplary embodiment.

FIG. 22B shows the MS2 mass spectrum of tryptic peptide product ions detected in an eluate from an NIST mAb direct digest subjected to a regular flow CSH LC column (bottom), and the MS2 mass spectrum of glycine to aspartic acid SV tryptic peptide product ions detected (SVA as low as 0.004%) in an eluate from a digested ProteoMiner™ NIST mAb subjected to a nanoLC column (top), according to an exemplary embodiment. Figure discloses SEQ ID NOS 25-26, respectively, in order of appearance.

FIG. 22C shows the number of NIST mAb serine, glycine, or valine sequence variations identified using eluates from NIST mAb direct digests subjected to regular flow CSH LC or nanoLC columns or digested ProteoMiner™ NIST mAb eluates subjected to nanoLC columns, according to an exemplary embodiment.

FIG. 22D shows the number of NIST mAb serine, glycine, or valine sequence variations identified by three labs using eluates from NIST mAb direct digests subjected to regular flow CSH LC columns or digested ProteoMiner™ NIST mAb eluates subjected to nanoLC columns, according to an exemplary embodiment.

FIG. 22E shows the NIST mAb alanine to threonine, glycine to aspartic acid, serine to asparagine, valine to leucine or isoleucine, arginine to lysine, and lysine to arginine sequence variations identified by three labs using eluates from NIST mAb direct digests subjected to regular flow CSH LC or nanoLC columns or digested ProteoMiner™ NIST mAb eluates subjected to nanoLC columns, according to an exemplary embodiment.

FIG. 23 shows unsaturated (bottom) and saturated (top) peaks in the MS2 mass spectrum of tryptic peptide product ions (e.g., VVSVLTVLHQDWLNGK (SEQ ID NO: 6) and TTPPVLDSDGSFEYSK (SEQ ID NO: 7)) and serine to asparagine SV tryptic peptide product ions (e.g., VVNVLTVLHQDWLNGK (SEQ ID NO: 8) and TTPPVLDSDGSFEYNK (SEQ ID NO: 9)) detected in an eluate from a digested ProteoMiner™ NIST mAb subjected to a nanoLC column, according to an exemplary embodiment.

FIG. 24 shows that analyzing an eluate from an NIST mAb direct digest subjected to a regular flow CSH LC column using a mass spectrometer produces larger peaks in the MS2 mass spectrum of tryptic peptide product ions (Scan 9602, z=3) than in the MS2 mass spectrum of cysteine to serine SV tryptic peptide product ions (Scan 9515, z=3), whereas analyzing an eluate from a ProteoMiner™ enriched NIST mAb digest subjected to a nanoLC column using a mass spectrometer produces smaller peaks in the MS2 mass spectrum of tryptic peptide product ions (Scan 59496, z=3) than in the MS2 mass spectrum of cysteine to serine SV tryptic peptide product ions (Scan 59579, z=3), according to an exemplary embodiment. Figure discloses SEQ ID NOS 27-28, and 27-28, respectively, in order of appearance.

FIG. 25 shows that a mass spectrometer does not generate y-ions in the MS2 mass spectrum of serine to leucine or isoleucine SV tryptic peptide product ions using an eluate from an NIST mAb direct digest subjected to a regular flow CSH LC column (Scan 14203, z=4), whereas a mass spectrometer generates y-ions in the MS2 mass spectrum of serine to leucine or isoleucine SV tryptic peptide product ions using an eluate from a digested ProteoMiner™ NIST mAb subjected to a nanoLC column (Scan 75616, z=4), according to an exemplary embodiment. Figure discloses “THTCPPCPAPELLGGPXVFLFPPKPK” as SEQ ID NO: 29.

### DETAILED DESCRIPTION

In order to manufacture biopharmaceutical products, it is important to obtain biopharmaceutical products having high purity, since residual HCPs can compromise product safety and stability. For producing cell-based recombinant therapeutic antibodies, typically, immuno-assays, such as enzyme-linked immunosorbent assays (ELISA), have been used to monitor HCP removal (clearance) using polyclonal anti-HCP antibodies during process development. ELISA can provide semi-quantitation of total HCP levels with high throughput. However, since polyclonal anti-HCP antibodies are used for ELISA to capture, detect and quantify total HCPs, they may not be effective in quantitating individual HCPs. In particular, some non-immunogenic or weakly-immunogenic HCPs may not be detected using ELISA.

In order to both identify and quantify HCPs, several complementary approaches have been used to monitor HCPs, such as one-dimensional/two-dimensional (1D/2D) PAGE or liquid chromatography (LC) coupled tandem mass spectrometry (LC-MS/MS). However, the wide dynamic concentration ranges of HCPs in the presence of high concentrations of purified antibodies may be a major challenge for developing LC-MS methods to monitor the removal of HCP impurities. Mass spectrometry (MS) alone lacks the capability to detect low abundance targets, such as low ppm levels of HCPs, in the presence of high concentrations of therapeutic antibodies due to the wide dynamic concentration ranges, which can be over six orders of magnitude higher than HCP impurities. To overcome this issue, one strategy can be to resolve the co-eluting peptides before MS analysis by adding another dimension of separation, such as 2D-LC and/or ion mobility, in combination with data-dependent acquisition or data-independent acquisition to increase the separation efficiency.

Huang et al. (Huang et al., A Novel Sample Preparation for Shotgun Proteomics Characterization of HCPs in Antibodies, Anal. Chem. 2017, May 16; 89 (10):5436-5444) describes a sample preparation method using trypsin digestion for shotgun proteomics characterization of HCP impurities in an antibody sample. Huang's sample preparation method maintains the antibody nearly intact while HCPs are digested. Huang's approach can reduce the dynamic range for HCP detection using mass spectrometry by one to two orders of magnitude compared to traditional trypsin digestion sample preparation. As demonstrated by HCP spiking experiments, Huang's approach can detect 0.5 ppm of HCPs with molecular weight greater than 60 kDa, such as rPLBL2. For example, sixty mouse HCP impurities were detected in RM 8670 (NISTmAb, NIST monoclonal antibody standard, expressed in a murine cell line, obtained from the National Institute of Standards and Technology, Gaithersburg, Md.) using Huang's approach.

Doneanu et al. (Doneanu et al., Enhanced Detection of Low-Abundance Host Cell Protein Impurities in High-Purity Monoclonal Antibodies Down to 1 ppm Using Ion Mobility Mass Spectrometry Coupled with Multidimensional Liquid Chromatography, Anal. Chem. 2015 Oct. 20; 87(20):10283-10291) reports the detection of low-abundance HCP impurities down to 1 ppm in antibody samples using liquid chromatography-mass spectrometry (LC-MS) methods. Doneanu's approach includes using a new charge-surface-modified C18 stationary phase to mitigate the challenges of column saturation, incorporating traveling-wave ion mobility separation of co-eluting peptide precursors, and improving fragmentation efficiency of low-abundance HCP peptides by correlating the collision energy used for precursor fragmentation with the mobility drift time. HCP impurities can be identified at 10-50 ppm using 2D-HPLC (2D-High Performance Liquid Chromatography) in combination with ion mobility mass spectrometry analysis. However, the cycle times for 2D-LC or 2D-HPLC can be very long. In addition, these methods may not be sensitive enough for low level HCP analysis, such as less than 10 ppm. Other approaches of identifying HCP impurities include sample preparations to enrich HCPs by removing antibodies in the sample, such as using affinity purification or limited digestion to remove antibodies. In addition, using polyclonal antibodies to capture HCPs is another common approach.

Analytical techniques required for identifying HCP impurities encounter the challenges of dealing with about 1 million times more matrix molecules than the analytes, for example, HCPs or HCP peptides, due to very high sample complexity. Enriching HCPs to levels compatible with detection is difficult, since HCP impurities are most often present at low levels, such as 1-100 ppm, in protein biopharmaceuticals. Without knowing the identities and properties of HCPs, it can be very challenging to develop a general sample preparation procedure to enrich HCPs (or HCP peptides) or remove the matrix background (Doneanu et al.).

Chen et al. (Chen et al., Improved host cell protein analysis in monoclonal antibody products through ProteoMiner, Anal. Biochem. 2020 Dec. 1; 610:113972) describes a method of enriching HCPs using interacting peptide ligands, particularly ProteoMiner™ beads. The method of the present invention improves upon the previously described ProteoMiner™ method of HCP enrichment, identification and quantification.

The present application provides a method to enrich HCPs using interacting peptide ligands, such as a combinatorial ligand library. In some exemplary embodiments, ProteoMiner™ beads (Bio-Rad Laboratories, Inc., Hercules, Calif.), a combinatorial hexapeptide library immobilized on beads, are used to enrich HCPs. When the peptide ligand-conjugated beads are applied to a sample containing various protein species, each protein species can bind to its interacting peptide ligands. HCPs bind to their interacting peptide ligands mainly by hydrophobic force in combination with some weak interaction forces, such as ionic interaction and hydrogen bonding.

A protein species that is in high abundance can saturate its interacting peptide ligands due to the presence of excess quantity, since there are limited numbers of interacting peptide ligands corresponding to each protein species in the combinatorial ligand library. The limited numbers of corresponding interacting peptide ligands can be saturated easily in the presence of excess quantity of high-abundance proteins. The excess quantity of high-abundance proteins that are unable to bind to the interacting peptide ligands can be washed off from the beads. Since the quantity of low-abundance proteins in the sample is relatively low in comparison to the high-abundance proteins, the low-abundance proteins may not saturate their corresponding interacting peptide ligands. Therefore, the low-abundance proteins can be relatively enriched in comparison to the high-abundance proteins. After conducting the enrichment process, the broad dynamic range of protein concentrations can be significantly reduced to allow detection of low abundance proteins.

The broad dynamic range of protein concentrations can be further reduced using limited digestion. Decreasing the ratio of digestive enzyme to substrate, and performing a digestion reaction on natively folded instead of denatured proteins, results in incomplete digestion of proteins in a sample, disproportionately reducing the presence of peptides corresponding to a high-abundance protein in the sample, and therefore decreasing the dynamic range of protein concentrations.

The HCP enrichment method of the present application can enrich and detect mid-abundance and low-abundance proteins by decreasing the quantity of high-abundance proteins. The HCP enrichment method of the present application also fulfills the need of enriching low abundance HCP impurities in drug products or other samples of interest.

In some exemplary embodiments, samples are treated with ProteoMiner™ beads to reduce the quantity of therapeutic proteins that are present in high abundance and to enrich low abundance HCP impurities. The HCP-enriched sample is subsequently subjected to proteomic analysis. This procedure can enrich the low abundance HCP impurities and reduce the levels of therapeutic protein at the same time. It can successfully reduce the dynamic concentration ranges among HCPs and protein drugs, allowing for the detection of low abundance HCP impurities. The detection limit of the HCP impurities using the HCP enrichment method of the present application is about 0.003-0.006 ppm.

In some exemplary embodiments, the present disclosure provides a method of identifying and/or quantifying host cell protein (HCP) impurities in a sample, comprising: contacting a sample including at least one high-abundance peptide or protein and at least one HCP impurity to a solid support, wherein said solid support is attached to interacting peptide ligands capable of interacting with said at least one HCP impurity; washing the solid support to provide an eluate comprising at least one enriched HCP impurity; subjecting the eluate to an enzymatic digestion condition to generate at least one component of the at least one enriched HCP impurity, wherein the enzymatic digestion condition is a limited digestion that does not fully digest all proteins in the eluate; identifying and/or quantifying the at least one component of the at least one enriched HCP impurity using a mass spectrometer; and using the identification and/or quantification of the at least one component to identify and/or quantify the at least one enrich HCP impurity.

In some exemplary embodiments, phase transfer surfactants (PTS), such as sodium deoxycholate (SDC) and sodium lauryl sulfate (SLS), are used to elute HCPs from ProteoMiner™ beads. SDC is an ionic detergent that is especially useful for disrupting and dissociating protein interactions. Ionic detergents have a hydrophilic head group that is charged and can be either negatively (anionic) or positively (cationic) charged. SLS is an anionic surfactant. Anionic detergents, such as SLS or sodium dodecylbenzene sulphonate, are sodium salts of sulphonated long chain, alcohols or hydrocarbons.

In some exemplary embodiments, the elution buffer to elute HCPs from ProteoMiner™ beads contains ionic surfactants, anionic surfactants, cationic surfactants, phase transfer surfactants, or combinations thereof. In one aspect, the elution buffer contains SDC, SLS, or sodium dodecylbenzene sulphonate. In one aspect, the elution buffer comprises PTS buffer containing 12 mM SDC (sodium deoxycholate), 12 mM SLS (sodium lauroyl sarcosinate), 10 mM TCEP (Tris(2-carboxyethyl)phosphine, a reducing agent) and 30 mM CAA (chloroacetamide).

Trace amounts of particular HCPs may cause immune response or toxic biologic activities after drug injection. The presence of residual HCPs in biopharmaceutical products is a concern for drug safety, which has led to an increasing demand for developing methods and systems to identify and characterize HCP impurities in biopharmaceutical products. There are unmet needs to identify and monitor individual HCPs for risk assessment in therapeutic protein products.

This disclosure provides methods and systems to satisfy the aforementioned demands by providing methods and systems to identify and quantitate HCPs to monitor and control the residual HCPs in drug substance to mitigate safety risks. Exemplary embodiments disclosed herein satisfy the aforementioned demands and the long felt needs.

In addition to HCPs, sequence variants (SVs) resulting from unintended amino acid substitutions are another product quality attribute of concern in drug development and manufacturing. Such SVs have been shown to exist in both natural and recombinant proteins, and are believed to be caused by a number of mechanisms including DNA mutations during replication, and transcriptional and translational errors during the protein biosynthesis process.

Due to the high fidelity of biologic systems, which evolved to prevent the occurrence of such spontaneous errors, the SVs are usually present at a very low level (<0.1%) in natural biologic proteins. However, during therapeutic protein drug development, the aim is to increase the protein titer and process productivity to meet global demand and reduce the cost of goods for expanded patient access. This has led to the wide use of the so-called intensified bioreactor manufacturing systems, which are designed to maximize cell density and specific productivity for the target therapeutic proteins during the cell culture process. Such intensified production systems can impose higher than normal expression machinery stress to the production cell lines. If not fully optimized, elevated levels of SVs could be generated in the protein products. In addition, to further increase the product titer, cell line development usually goes through multiple rounds of selection with increasing selective stresses to find the top-producing cell clone. This selection process could potentially introduce DNA mutations to the cell lines. If not properly screened, it could lead to unexpectedly high levels of SVs in the final drug products.

Given these concerns regarding how elevated SVs might affect drug quality, both the industry and regulatory agencies have started to pay more attention to SVs. Over the past decade, substantial efforts and resources have been invested across the industry to better understand the causes of SVs and their control in biologic development. As a result of these collective efforts, several control strategies have been developed to best monitor and mitigate the SV issue during their product and process development. As expected, these proposed strategies highlighted the importance of a multi-assay, multi-tier SV screening approach to guide process development from early cell line selection to small-scale cell culture process development to scale-up confirmation. Together, these strategies have provided a valuable and industry-wide framework and high-level guidance toward the goal of establishing some common best practices in terms of SV control.

However, there is still a lack of clarity and consensus across the industry on a variety of important aspects. These include, for example: 1) the selection and combinatory use of multiple SV-relevant analytical technologies (e.g., next-generation sequencing-based DNA or RNA sequencing, liquid chromatography (LC)-mass spectrometry (MS)/MS, surrogate amino acid analysis); 2) the selection of stage(s) and degree to implement SV monitoring and control during the product and process development considering both overall control strategy effectiveness and development timeline; 3) the appropriate assessment of SV risk on product safety and efficacy; 4) determination of a rational SV control limit or acceptable level in process development and in the final drug products; and 5) reporting of the SV data in regulatory filing.

To fill some of these knowledge gaps, the results of a survey of industry practices on SV analysis and control in their biologic development were published recently by the International Consortium for Innovation & Quality in Pharmaceutical Development. (Zhang, et al. 2020). In the survey, one of the most critical questions asked is the level of SVs that individual companies set as an action limit (or control target) for their product and process development. Problematically, there isn't a reliable method for reproducibly detecting the same set of SV mAbs within a sample. For example, a previous study evaluated the performance of an LC-MS method for SV NIST mAb detection because it was well characterized and two independent laboratories had performed a similar analysis. (Zhang, et al. 2020). Although all three laboratories were able to detect and identify low-level SVs in the range of 0.01-0.1%, the sets of SVs identified by the three laboratories did not completely overlap with each other. The three testing laboratories each identified a similar number (e.g., 21-23) of SVs in the NIST mAb, but only 12 of them were commonly identified by all three testing laboratories, suggesting that there is a large method-based variation in detecting low-level SVs.

The present application provides methods to enhance the detection limit of SV proteins, particularly mAbs, with or without enrichment using interacting peptide ligands, such as a combinatorial ligand library. In some exemplary embodiments, ProteoMiner™ beads (Bio-Rad Laboratories, Inc., Hercules, Calif.), a combinatorial hexapeptide library immobilized on beads, are used to improve the detection limit of SV mAbs (e.g., resolution at which SV mAbs can be detected). In some exemplary embodiments, ProteoMiner™ beads can enrich SV mAbs in which an amino acid substitution effects the mAb protein structure. When the peptide ligand-conjugated beads are applied to a sample containing various protein species, each protein species can bind to its interacting peptide ligands. SV proteins bind to their interacting peptide ligands mainly by hydrophobic force in combination with some weak interaction forces, such as ionic interaction and hydrogen bonding.

A high-abundance non-SV protein species and its corresponding low-abundance SV protein species may bind the same interacting peptide ligands. The affinity of a low-abundance SV protein species for a peptide ligand may equal the affinity of the corresponding non-SV protein species for the same peptide ligand. Alternatively, the affinity of a low-abundance SV protein species for a peptide ligand may be greater or less than the affinity of the corresponding non-SV protein species for the same peptide ligand. The excess quantity of high-abundance non-SV proteins that are unable to bind to the interacting peptide ligands can be washed off from the beads. Therefore, the detection limit of the low-abundance SV protein species can be relatively improved in comparison to the high-abundance non-SV protein species. After improving the detection limit of the low-abundance SV protein species, the broad dynamic range of protein concentrations can be significantly reduced to allow detection of low-abundance SV proteins.

The broad dynamic range of protein concentrations may be further reduced using limited digestion. Decreasing the ratio of digestive enzyme to substrate, and performing a digestion reaction on natively folded instead of denatured proteins, results in incomplete digestion of proteins in a sample, which may disproportionately reduce the presence of peptides corresponding to a high-abundance protein in the sample, and decrease the dynamic range of protein concentrations.

The detection limit of low-abundance SV protein species can be further enhanced using nanoflow LC (nanoLC). NanoLC can improve the MS2 spectra by increasing the signal of SV peptide product ions that derive from SV proteins and allowing the formation of more y-ions.

The enhanced SV protein detection method of the present application can enhance the detection limit of SV proteins by decreasing the quantity of high-abundance non-SV proteins. The method of enhancing the detection limit of SV proteins of the present application can also fulfill the need of enriching low-abundance SV proteins in therapeutic drug products.

In some exemplary embodiments, samples are treated with ProteoMiner™ beads to reduce the quantity of therapeutic proteins that are present in high abundance and to enhance the detection of low-abundance SV therapeutic proteins with or without enrichment. Samples are subsequently subjected to proteomic analysis. This procedure can enrich the low-abundance SV therapeutic proteins and reduce the levels of non-SV therapeutic protein at the same time. It can successfully reduce the dynamic concentration ranges among SV and non-SV protein drugs, allowing for the detection of low-abundance SV proteins. The enhanced SV protein detection method of the present application can detect an amino acid substitution that occurs in about 0.003% of proteins.

In some exemplary embodiments, the present disclosure provides a method of identifying sequence variant (SV) peptides or proteins in a sample, wherein at least one amino acid of a SV peptide or protein unintentionally differs from a wild-type peptide or protein, comprising: (a) contacting a sample including at least one more-abundant wild-type peptide or protein and at least one SV peptide or protein to a solid support, wherein said solid support is attached to interacting peptide ligands capable of interacting with said at least one SV peptide or protein; (b) washing said solid support to provide a first eluate comprising at least one enriched SV peptide or protein; (c) subjecting said first eluate to an enzymatic digestion condition to generate at least one component of said at least one enriched SV peptide or protein; (d) subjecting said first eluate with said at least one component of said at least one enriched SV peptide or protein to a liquid chromatography system to produce a second eluate with said at least one component of said at least one enriched SV peptide or protein; (e) subjecting said second eluate with said at least one component of said at least one enriched SV peptide or protein to mass spectrometry; (f) identifying said at least one component of said at least one enriched SV peptide or protein using a mass spectrometer; and (g) using the identification of said at least one component of said at least one enriched SV peptide or protein to identify said at least one enriched SV peptide or protein in said sample.

In some exemplary embodiments, phase transfer surfactants (PTS) are used to elute SV proteins from ProteoMiner™ beads. In some exemplary embodiments, the elution buffer to elute SV proteins from ProteoMiner™ beads contains ionic surfactants, anionic surfactants, cationic surfactants, phase transfer surfactants, or combinations thereof. In one aspect, the elution buffer contains SDC, SLS, or sodium dodecylbenzene sulphonate. In one aspect, the elution buffer comprises PTS buffer containing 12 mM SDC (sodium deoxycholate), 12 mM SLS (sodium lauroyl sarcosinate), 10 mM TCEP (Tris(2-carboxyethyl)phosphine, a reducing agent) and 30 mM CAA (chloroacetamide).

Trace amounts of particular SV proteins may cause immune response or toxic biologic activities after drug injection. The presence of SV proteins in biopharmaceutical products has been a concern for drug safety, which has led to an increasing demand for developing methods and systems to identify and characterize SV proteins in biopharmaceutical products. There are unmet needs to identify and monitor SV proteins for risk assessment of the presence of SV proteins in therapeutic protein products.

This disclosure provides methods and systems to satisfy the aforementioned demands by providing methods and systems to identify and quantitate SV proteins in order to monitor and control the SV proteins in drug substance to mitigate safety risks. Exemplary embodiments disclosed herein satisfy the aforementioned demands and the long-felt needs.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule.

As used herein, the term “therapeutic protein” includes any of proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 BIOTECHNOLOGY AND GENETIC ENGINEERING REVIEWS 147-176 (2012), the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In one aspect, the at least one high-abundance peptide or protein in the method of the present invention is an antibody, a bispecific antibody, an antibody fragment, a Fab region of an antibody, an antibody-drug conjugate, a fusion protein, a protein pharmaceutical product, or a drug.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CHL CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the systems and methods disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

As used herein, the term “host-cell protein” (HCP) includes protein derived from the host cell. Host-cell protein can be a process-related impurity which can be derived from the manufacturing process and can include three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables. In some exemplary embodiments, the types of HCP process-related impurities in the composition can be at least two.

In some exemplary embodiments, a sample can comprise at least one high-abundance protein or peptide and at least one HCP. In some exemplary embodiments, a concentration of the at least one high-abundance protein or peptide can be at least about 1000 times, about 10,000 times, about 100,000 times or about 1,000,000 times higher than a concentration of the at least one HCP. Another way of expressing the relative concentrations is, for example, in parts per million (ppm). It should be understood that when using ppm to describe the concentration of a low-abundance protein or peptide, such as an HCP, in a sample that includes a high-abundance protein or peptide, such as a therapeutic protein, ppm is measured relative to the concentration of the high-abundance protein or peptide. In some exemplary embodiments, a concentration of the at least one HCP can be less than about 1000 ppm, less than about 100 ppm, less than about 10 ppm, or less than about 1 ppm.

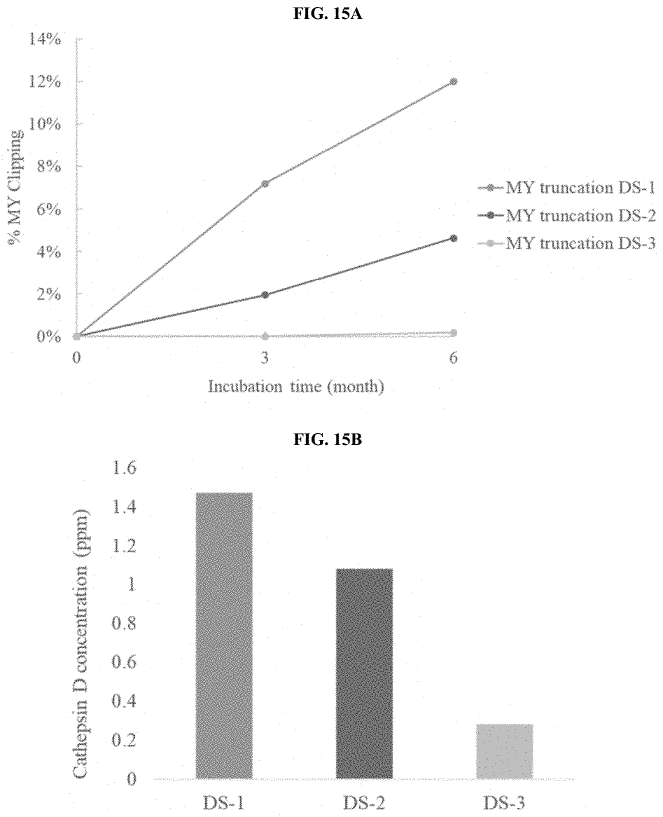

As used herein, the term “sequence variant protein” (SV protein) includes any protein with an unintentionally substituted amino acid. For example, unintentional amino acid substitutions within SV proteins can result from at least one DNA mutation in the coding sequence, transcriptional error from DNA to mRNA, translational error from mRNA to protein sequence, or a combination thereof, as seen in FIG. 15. As reviewed in literature, due to the finite fidelity in the DNA replication and protein biosynthesis process, unintended amino acid substitution occurs naturally in any natural biologic system in a spontaneous manner. However, in normal biologic systems, the chance for such spontaneous errors to occur is expected to be extremely low, with the range of 10⁻¹¹-10⁻⁸ during DNA replication, 10⁻⁶-10⁻⁴ during mRNA transcription and 10⁻⁵-10⁻⁴ during protein translation. In prokaryotic systems, like E. coli, the translational error could be higher, up to 10⁻³, or 0.1% of SVs relative to its native form. Due to the spontaneous nature of the events, these very low levels of SVs resulting from transcriptional or translational errors are usually inevitable, and thus can be considered as the biologic noise in protein expression. Under-optimized recombinant protein production systems may elevate SV proteins, for example, by containing a rare codon sequence or as a result of depletion of an amino acid.

In some exemplary embodiments, a sample can comprise at least one high-abundance non-SV protein or peptide and at least one SV protein. In some exemplary embodiments, a concentration of the at least one high-abundance non-SV protein or peptide can be at least about 1000 times, about 10,000 times, about 100,000 times or about 1,000,000 times higher than a concentration of the at least one SV protein. Another way of expressing the relative concentrations is, for example, in parts per million (ppm). It should be understood that when using ppm to describe the concentration of a low-abundance protein or peptide, such as a SV protein, in a sample that includes a high-abundance non-SV protein or peptide, such as a therapeutic protein, ppm is measured relative to the concentration of the high-abundance non-SV protein or peptide. In some exemplary embodiments, a concentration of the at least one SV protein can be less than about 1000 ppm (e.g., 0.1%), less than about 100 ppm (e.g., 0.01%), less than about 10 ppm (e.g., 0.001%), or less than about 1 ppm (e.g., 0.0001%).

While the present disclosure primarily concerns HCPs and SVs, it should be understood that the methods and systems of the present invention can be used for identification and quantification of any low-abundance peptides or proteins in a sample.

As used herein, a “protein pharmaceutical product” or “biopharmaceutical product” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

As used herein, a “sample” can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration. In some specific exemplary embodiments, the drug product can be selected from manufactured drug product in the clinic, shipping, storage, or handling.

As used herein, the term “solid support” can include any surface with an ability to bind a protein or peptide. Non-limiting examples of solid supports can include affinity resins, beads and coated plates or microplates. Solid supports can be attached to molecules capable of binding to a protein or peptide, including affinity reagents, antigen-binding molecules, or interacting peptide ligands. In some exemplary embodiments, a solid support comprises beads attached to interacting peptide ligands. In some exemplary embodiments, a solid support comprises ProteoMiner™ beads.

In some exemplary embodiments, the sample can be prepared prior to LC-MS analysis. Preparation steps can include denaturation, alkylation, dilution and digestion.

As used herein, the term “protein alkylating agent” or “alkylation agent” refers to an agent used for alkylating certain free amino acid residues in a protein. Non-limiting examples of protein alkylating agents are iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, 12 JOURNAL OF PROTEOME RESEARCH 1067-1077 (2013)).

Conventional methods use a digestive enzyme in conditions and concentrations sufficient to completely digest all protein in a sample prior to LC-MS analysis. The present disclosure surprisingly finds that identification and quantification of low-abundance proteins such as HCPs can be improved through limited digestion, meaning that digestive enzymes are used in conditions such that proteins in a sample are not completely digested. In some exemplary embodiments, proteins are subjected to digestion without prior denaturation, meaning that “native digestion” is conducted on natively folded proteins. In some exemplary embodiments, a ratio of digestive enzyme to substrate is selected to ensure limited digestion. In some exemplary embodiments, a ratio of digestive enzyme to substrate is less than about 1:100, less than about 1:200, less than about 1:300, less than about 1:400, less than about 1:500, less than about 1:600, less than about 1:700, less than about 1:800, less than about 1:900, less than about 1:1000, less than about 1:2000, less than about 1:3000, less than about 1:4000, less than about 1:5000, less than about 1:6000, less than about 1:7000, less than about 1:8000, less than about 1:9000, less than about 1:10000, about 1:400, about 1:1000, about 1:2500, or about 1:10000.

As used herein, the term “protein reducing agent” or “reduction agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

As used herein, the term “liquid chromatography” refers to a process in which a biological and/or chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, the sample or eluate can be subjected to any one of the aforementioned chromatographic methods or a combination thereof.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

The mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

SRM/MRM/Selected-ion monitoring (SIM) is a method used in tandem mass spectrometry in which an ion of a particular mass is selected in the first stage of a tandem mass spectrometer and an ion product of a fragmentation reaction of the precursor ion is selected in the second mass spectrometer stage for detection. Examples of triple quadrupole mass spectrometers (TQMS) that can perform MRM/SRM/SIM include but are not limited to QTRAP® 6500 System (Sciex), QTRAP® 5500 System (Sciex), Triple QTriple Quad 6500 System (Sciex), Agilent 6400 Series Triple Quadrupole LC/MS systems, and Thermo Scientific™ TSQ™ Triple Quadrupole system.

In addition to MRM, the choice of peptides can also be quantified through Parallel-Reaction Monitoring (PRM). PRM is the application of SRM with parallel detection of all transitions in a single analysis using a high-resolution mass spectrometer. PRM provides high selectivity, high sensitivity and high-throughput to quantify selected peptide (Q1), hence quantify proteins. Multiple peptides can be specifically selected for each protein. PRM methodology can use the quadrupole of a mass spectrometer to isolate a target precursor ion, fragment the targeted precursor ion in the collision cell, and then detect the resulting product ions in the Orbitrap mass analyzer. PRM can use a quadrupole time-of-flight (QTOF) or hybrid quadrupole-orbitrap (QOrbitrap) mass spectrometer to carry out the identification of peptides and/or proteins. Examples of QTOF include but are not limited to TripleTOF® 6600 System (Sciex), TripleTOF® 5600 System (Sciex), X500R QTOF System (Sciex), 6500 Series Accurate-Mass Quadrupole Time-of-Flight (Q-TOF) (Agilent) and Xevo G2-XS QT of Quadrupole Time-of-Flight Mass Spectrometry (Waters). Examples of QObitrap include but are not limited to Q Exactive™ Hybrid Quadrupole-Orbitrap Mass Spectrometer (Thermo Scientific) and Orbitrap Fusion™ Tribrid™ (Thermo Scientific).

Non-limiting advantages of PRM include: elimination of most interferences; provides more accuracy and attomole-level limits of detection and quantification; enables the confident confirmation of the peptide identity with spectral library matching; reduces assay development time since no target transitions need to be preselected; and ensures UHPLC-compatible data acquisition speeds with spectrum multiplexing and advanced signal processing.

The mass spectrometer in the methods or systems of the present application can be, for example, an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-PRM-MS (liquid chromatography-parallel reaction monitoring-mass spectrometry) analyses. In some exemplary embodiments, the identification of peptides is performed using PRM-MS.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS2, can be performed by first selecting and isolating a precursor ion (MS1), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com/proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

It is understood that the present invention is not limited to any of the aforesaid protein(s), therapeutic protein(s), antibody(s), recombinant protein(s), host-cell protein(s), sequence variant protein(s), protein pharmaceutical product(s), sample(s), solid support(s), protein alkylating agent(s), protein denaturing agent(s), protein reducing agent(s), digestive enzyme(s), chromatographic method(s), mass spectrometer(s), database(s), bioinformatics tool(s), pH range(s) or value(s), temperature(s), or concentration(s), and any protein(s), therapeutic protein(s), antibody(s), recombinant protein(s), host-cell protein(s), sequence variant protein(s), protein pharmaceutical product(s), sample(s), solid support(s), protein alkylating agent(s), protein denaturing agent(s), protein reducing agent(s), digestive enzyme(s), chromatographic method(s), mass spectrometer(s), database(s), bioinformatics tool(s), pH, temperature(s), or concentration(s) can be selected by any suitable means.

The present invention will be more fully understood by reference to the following examples. They should not, however, be construed as limiting the scope of the invention.

### EXAMPLES

### Materials and Methods for Examples 1-3

### Materials

ProteoMiner™ Protein Enrichment kit was purchased from Bio-Rad Laboratories, Inc. (Hercules, Calif.). ProteoMiner™ technology is a sample preparation tool for the compression of the dynamic range of protein concentration in biological samples. A large library of combinatorial hexapeptide ligands were immobilized on beads for capturing various proteins. ProteoMiner™ spin column contained 500 μl bead slurry (4% beads, 20% v/v aqueous EtOH) with 20 μl settled bead volume. The wash buffer of the kit contains 50 mL PBS (phosphate-buffer saline, 150 mM NaCl, 10 mN NaH2PO4, pH 7.4). The elution buffer of the kit contains lyophilized urea CHAPS (8 M urea, 2% CHAPS; CHAPS detergent is 3-((3-cholamidopropyl) dimethylammonio)-1-propanesulfonate). The rehydration buffer of the kit contains 5% acetic acid.

Chromatography solvents which were LC-MS grade were purchased from Thermo Fisher Scientific (Waltham, Mass.). Monoclonal antibodies were produced by Regeneron (Tarrytown, N.Y.). Sodium deoxycholate (SDC), sodium lauroyl sarcosinate (SLS) and chloroacetamide (CAA) were purchased from Sigma-Aldrich (St. Louis, Mo.). Tris-(2-carboxyethyl) phosphine (TCEP) was purchased from Thermo Fisher Scientific. RM 8670 (NISTmAb, NIST monoclonal antibody standard, expressed in a murine cell line) was obtained from the National Institute of Standards and Technology (NIST, Gaithersburg, Md.).

### Protein Enrichments Using ProteoMiner™ Protein Enrichment Kit

ProteoMiner™ Protein Enrichment kit was used to enrich proteins in samples. A small-scale ProteoMiner™ cartridge was used for five experiments. ProteoMiner™ beads were washed twice with 200 μL of washing buffer provided in the kit. The beads were resuspended with 200 μL of water and 40 μL of beads slurry was transferred to a tube for conducting one experiment. mAB DS or NISTmAb was diluted with water followed by adjusting pH of the solution to pH 6 using 25 mM pH 4.0 sodium acetate. The sample was added to ProteoMiner™ bead slurry for incubation at room temperature with rotation for two hours. The sample mixture was then loaded into a tip with frit. The supernatant was removed by centrifuging at 1000×g for 1 minute. Subsequently, the beads were washed by adding 100 μL washing buffer into tip followed by centrifugation at 200×g for one minute three times. Finally, the enriched proteins were eluted using 10 μL of PTS buffer (12 mM SDC, 12 mM SLS, 10 mM TCEP and 30 mM CAA) followed by centrifugation at 200×g for one minute three times.

For an optimized ProteoMiner limited digestion method of the present invention, the collected eluate containing the enriched proteins was reduced. Reduced proteins were digested at an enzyme to substrate ratio of 1:400, at 28° C. overnight to obtain a solution containing a peptide mixture. The peptide mixture was then subjected to reduction, denaturation and alkylation.

The solution containing the peptide mixture was acidified using 10 μL of 10% TFA to precipitate SDC and SLS. Subsequently, the solution containing the peptide mixture was centrifuged at 14,000 rcf for twenty minutes. The supernatant containing the peptide mixture was then desalted using GL-Tip GC desalting tip, and dried using SpeedVac.

### LC-MS/MS Analysis

The desalted peptide mixture obtained from ProteoMiner™ limited digestion enrichment was dried and resuspended in 30 μL of 0.1% formic acid (FA) solution. Five μL of the solution containing the peptide mixture was injected into a low flow liquid chromatography system, for example, UltiMate™ 3000 RSLCnano system (Thermo Fisher Scientific) coupled to a Q-Exactive HFX mass spectrometer (Thermo Fisher Scientific). Peptides were separated on a 25 cm C18 column (inner diameter 0.075 mm, 2.0 μm, 100 Å, Thermo Fisher Scientific). The mobile phase buffer contained 0.1% FA in ultra-pure water (Buffer A) and the elution buffer contained 0.1% FA in 80% acetonitrile (ACN) (Buffer B). Peptides were eluted using a 100-minute linear gradient from 2-25% Buffer B at a flow rate of 300 nL/minute. The mass spectrometer was operated in data-dependent mode. The ten most intense ions were subjected to higher-energy collisional dissociation (HCD) fragmentation with the normalized collision energy (NCE) at 27% for each full MS scan at 120,000 resolution (automatic gain control (AGC) target 3e6, 60 ms maximum injection time, m/z 375-1500), and MS/MS events at 30,000 resolution (AGC target 1e5, 60 ms maximum injection time, m/z 200-2000). The MS proteomic data were deposited to the ProteomeXchange Consortium with project accession no. PXD016194 via JPOST repository.

### Example 1. Comparison of Digestion Methods

The previously described ProteoMiner™ method for HCP identification (Chen et al.) (the “direct digestion method”) was compared with various alternative techniques to optimize detection of host cell proteins (HCPs) and other low-abundance proteins in a sample comprising at least one high-abundance protein or peptide. The direct digestion method includes the steps of contacting a sample comprising at least one high-abundance protein or peptide to a solid support, such as beads, wherein interacting peptide ligands have been attached to the solid support and HCP impurities can bind to the interacting peptide ligands, for example ProteoMiner™ beads; washing the solid support using a solution comprising a surfactant to enrich HCP impurities and provide an eluate; subjecting the eluate to denaturation, alkylation and reduction; subjecting the denatured, alkylated and reduced eluate to an enzymatic digestion reaction to generate components of the enriched HCP impurities; identifying the components of the enriched HCP impurities using a mass spectrometer; and using the identified components to identify the enriched HCP impurities.

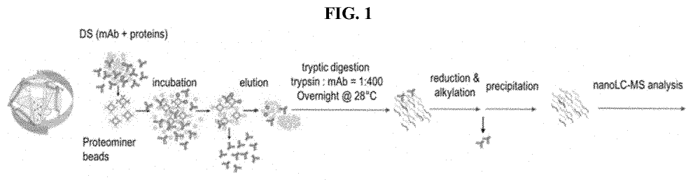

An alternative approach is an on-beads native digestion method, wherein HCP impurities are subjected to enzymatic digestion prior to elution from the solid support. Another alternative approach is “elution mild denatured digestion,” or “limited digestion,” wherein HCP impurities are eluted; subjected to limited digestion, using a lower ratio of digestive enzyme to substrate; subjected to reduction, denaturation and alkylation; and then analyzed using a mass spectrometer. An exemplary workflow of the limited digestion method is shown in FIG. 1.

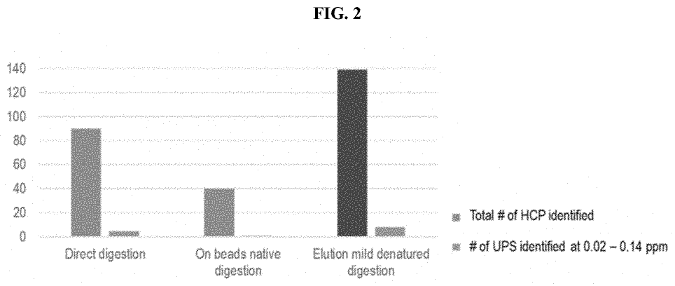

These three alternative techniques were compared based on their ability to identify HCPs in a monoclonal antibody drug substance (mAb DS) sample, and to identify spiked-in UPS2 proteins in mAb DS sample. UPS2 is a commercially available proteomics standard comprising 48 human proteins at a wide dynamic range of concentrations spanning many orders of magnitude. The ProteoMiner™ limited digestion was the most sensitive approach, as shown in Table 1 and FIG. 2.

TABLE 1
Comparison of digestion methods

| | | | | | Number |
|---|---|---|---|---|---|
| ProteoMiner + | Number of | | | | of UPS2 |
| Digestion | Host Cell | 2.14-16.58 | 0.16-1.5 | 0.02-0.14 | 0.02-0.14 |
| Method | Proteins | ppm | ppm | ppm | ppm |
| Direct Digestion | 90 | 8/8 | 8/8 | 5/8 | 5 |
| On Beads Native | 40 | 8/8 | 6/8 | 1/8 | 1 |
| Digestion | | | | | |
| Elution Limited | 139 | 8/8 | 7/8 | 8/8 | 8 |
| Digestion | | | | | |

### Example 2. Parameter Optimization

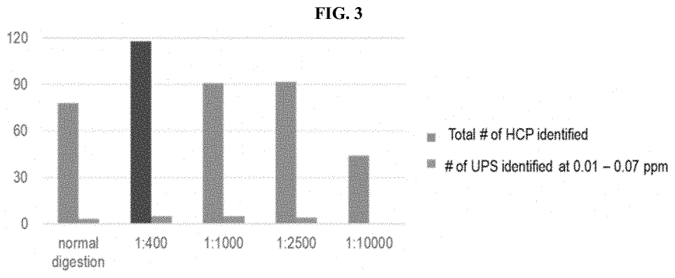

Based on the effectiveness of the limited digestion ProteoMiner™ method for low-abundance protein identification as shown in Example 1, this method was further optimized. The method was conducted with decreasing ratios of trypsin enzyme:substrate for digestion, and the sensitivity of HCP and UPS2 protein identification was compared. The previously described method used a ratio of 1:20 enzyme:substrate. A ratio of 1:400 enzyme:substrate was found to be the most effective, as shown in Table 2 and FIG. 3.

TABLE 2
Comparison of digestive enzyme:substrate ratios

| mAb | Number | | | | Number |
|---|---|---|---|---|---|
| trypsin | of Host | 1.07-8.29 | 0.08-0.75 | 0.01-0.07 | of UPS2 |
| Ratio | Cell Proteins | ppm | ppm | ppm | 0.01-0.07 ppm |
| 1:20 | 78 | 8/8 | 8/8 | 3/8 | 3 |
| 1:400 | 118 | 8/8 | 7/8 | 5/8 | 5 |
| 1:1000 | 91 | 8/8 | 7/8 | 5/8 | 5 |
| 1:2500 | 92 | 8/8 | 7/8 | 4/8 | 4 |
| 1:10000 | 44 | 8/8 | 7/8 | 0/8 | 0 |

While not being bound by theory, it is believed that more limited digestion may lead to a disproportional decrease in digestion of the high-abundance protein or proteins in the sample, further reducing the dynamic range of the digested peptides and allowing for more effective measurement of low-abundance proteins. Subjecting a protein sample to native digestion, without denaturation, contributes to more limited digestion, as does subjecting a protein sample to a lower ratio of digestive enzyme to substrate (Huang et al.).

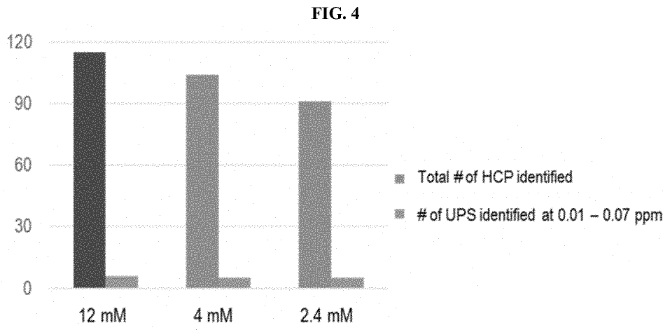

The method of the present invention was further optimized by comparing a range of concentrations of denaturation reagents. SLS/SDC concentration of 12 mM was found to be the most effective for HCP and UPS2 protein identification, as shown in Table 3 and FIG. 4.

TABLE 3
Comparison of denaturation agent concentrations

| | Number | | | | Number |
|---|---|---|---|---|---|
| | of Host | | | | of UPS2 |
| SLS/SDC | Cell | 1.07-8.29 | 0.08-0.75 | 0.01-0.07 | 0.01-0.07 |
| Concentration | Proteins | ppm | ppm | ppm | ppm |
| 12 mM | 115 | 8/8 | 7/8 | 6/8 | 6 |
| 4 mM | 104 | 8/8 | 7/8 | 5/8 | 5 |
| 2.4 mM | 91 | 8/8 | 7/8 | 5/8 | 5 |

These optimized conditions were used for further experiments.

### Example 3. Case Studies with an Optimized ProteoMiner™ Method

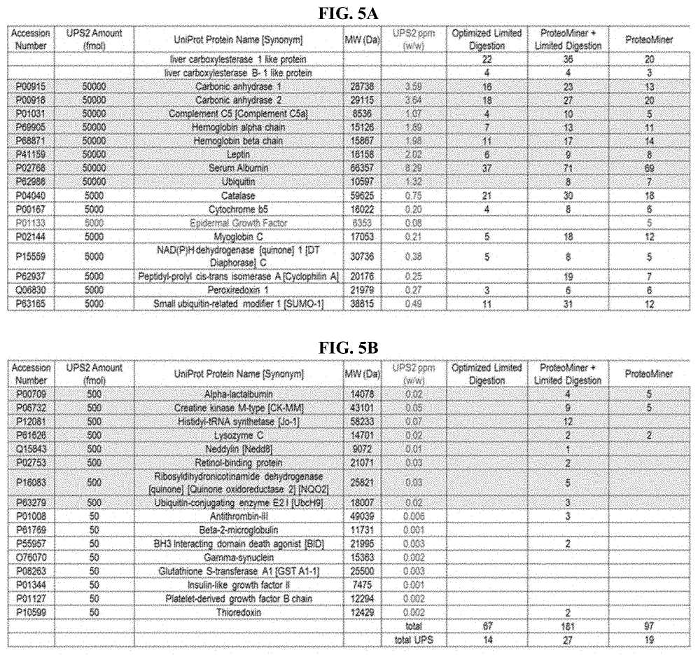

The optimized method described in Example 2 was used with UPS2 spiked into a mAb DS sample, compared to the previously described ProteoMiner™ method. The optimized method of the present invention had superior effectiveness at identifying low abundance UPS2 proteins compared to the previously described method, as shown in FIG. 5A and FIG. 5B. The UPS2 column on the right in Tables 1-3 represents directly digested UPS2 standard without being spiked into mAb DS, as a control for the detection limit of the instrument.

Both methods identified all spiked-in proteins at the 1-4 ppm level. At the 0.1-1 ppm level, the previously described ProteoMiner™ method identified 8/8 spiked-in proteins while the optimized ProteoMiner™ limited digestion method identified 7/8. At the 0.01-0.07 ppm level, the previously described ProteoMiner™ method identified 3/8 spiked-in proteins, while the optimized ProteoMiner™ limited digestion method identified 8/8. At the 0.001-0.006 ppm level, the previously described ProteoMiner™ method identified 0/8 spiked-in proteins, while the optimized ProteoMiner™ limited digestion method identified 3/8.

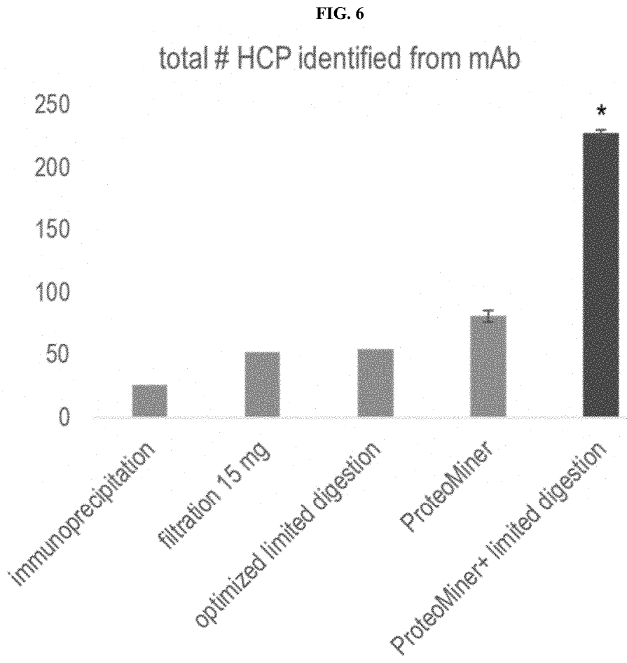

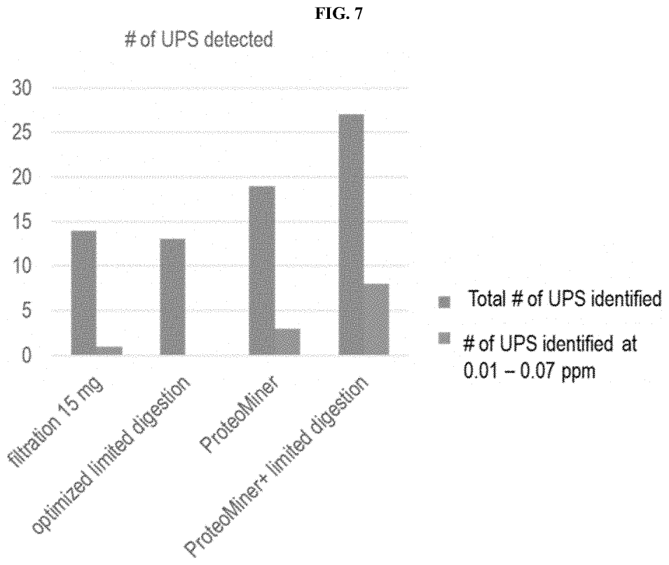

The optimized method of the present invention was further compared to conventional methods using mAb DS as a sample, with and without spiked-in UPS2. The conventional methods compared include immunoprecipitation, filtration, limited digestion alone, and the previously described ProteoMiner™ method. In mAb DS without spiked-in UPS2, the optimized method of the present invention was more effective at identifying HCPs than any other method, as shown in FIG. 6. In mAb DS with spiked-in UPS2, the optimized method of the present invention was more effective at identifying UPS2 proteins than any other method, as shown in FIG. 7. In FIG. 7, the first column represents the total number of UPS2 proteins identified, and the second column represents the number of UPS2 proteins identified at 0.1875 ppm, out of a total of eight. The optimized method of the present invention identified 8/8 UPS2 proteins at this concentration.

The optimized method of the present invention was further compared to previously described methods using NIST mAb standard as a sample. The conventional methods compared include normal digestion, native digestion, native digestion with ProA beads and field asymmetric ion mobility spectrometry (FAIMS), filtration, and the previously described ProteoMiner™ method. The number of HCPs identified in a NIST mAb sample using the method of the present invention was compared to the number of HCPs identified using conventional methods according to previous publications. The method of the present invention was more effective at identifying HCPs in the NIST mAb sample than any previously published method, as shown in FIG. 8.

### Materials and Methods for Examples 4-6

### Materials

Chromatography solvents were of LC-MS grade and were purchased from Thermo Fisher Scientific (Waltham, Mass.). The mAbs and spiked-in CHO proteins were produced by Regeneron (Tarrytown, N.Y.). Sodium deoxycholate (SDC), sodium lauroyl sarcosinate (SLS), iodoacetamide, urea, 10×Tris buffered saline, ammonium acetate, oleic acid and oleic acid-¹³C₁₈ (CAS number 287100-82-7) were purchased from Sigma-Aldrich (St. Louis, Mo.). Super-refined PS80 was purchased from Croda (East Yorkshire, UK). Dithiothreitol was purchased from Thermo Fisher Scientific. Human palmitoyl-protein thioesterase 1 (hPPT1) and human LPL were purchased from Abcam. CHO LAL, CHO complement component 1r (C1r-A), CHO acid ceramidase (ASAH1), CHO beta-2-microglobulin, CHO carboxypeptidase, CHO cathepsin D and CHO cathepsin Z were synthesized in-house by Regeneron Pharmaceuticals.

### Internal Standard and Standard Curve Preparation

Eight recombinant proteins (LAL, LPL, C1r-A, ASAH1, beta-2-microglobulin, carboxypeptidase, cathepsin D and cathepsin Z) were dissolved in water to a final concentration of 100 ng/μL as stock solutions. The stock solutions were further diluted to 1 ng/μL and 10 ng/μL, and spiked into an antibody matrix (mAb-1) for the preparation of standard curves with the following concentrations: 0.05 ppm, 0.1 ppm, 0.5 ppm, 1 ppm, 2 ppm and 5 ppm. The QC proteins were prepared from a separate stock of recombinant protein mixture (7 ng/μL cathepsin Z, 17 ng/μL cathepsin D, 35 ng/μL LAL, 87 ng/μL carboxypeptidase and 175 ng/μL LPL) and spiked into mAb-1 to obtain 0.2 ppm cathepsin Z, 0.5 ppm cathepsin D, 1 ppm LAL, 2.5 ppm carboxypeptidase and 5 ppm LPL. Heavy isotope labeled putative phospholipase B-like 2 (hPLBD2) was diluted to 5 ng/μL and spiked into each sample at 5 ppm.

Stock solutions of two recombinant proteins (LAL and hPPT1) were prepared at 1 ng/μL and 5 ng/μL and spiked into antibody matrix (mAb-2) for preparation of a standard curve with the following concentrations: 0.1 ppm, 0.5 ppm, 1 ppm, 2 ppm, 5 ppm, 10 ppm and 20 ppm. Heavy isotope labeled hPLBD2 was diluted to 5 ng/μL and spiked into each sample at 5 ppm. The same antibody matrix was used to measure the PPT-1 and LAL in mAb-18 and mAb-19.

Sample Preparation with the PMLD Method

Host cell proteins were first enriched through ProteoMiner enrichment coupled with limited digestion (PMLD). ProteoMiner beads were washed with wash buffer and water sequentially, then suspended in water. A total of 15 mg mAb was diluted or concentrated to 50 mg/mL in water, adjusted to pH 6 and added into the ProteoMiner bead slurry. Each sample was incubated with rotation at room temperature for 2.5 hours, then loaded onto an in-house made tip with a 9.5 mm pore size frit. The beads were then washed, and enriched proteins were eluted three times by addition of 10 μL of elution buffer (12 mM SDC and 12 mM SLS). The collected eluate was then further prepared through a modified limited digestion by addition of 75 ng trypsin, then digested at 28° C. overnight. The digested samples were reduced at 90° C. for 20 min and alkylated at room temperature for additional 20 min. The peptide mixture was acidified to pH 2-3 with 10% TFA to precipitate mAb, SDC and SLS in acidic solution. The mixture was then centrifuged at 14,000 rcf for 10 min. The peptide-containing supernatant was collected and desalted with GL-Tip GC desalting tips, dried and resuspended in 0.1% FA for nano-LC-MS/MS analysis.

Untargeted nanoLC-MS/MS and Targeted Parallel Reaction Monitoring Analysis

The peptide mixture was injected into an UltiMate™ 3000 RSLCnano system coupled to an Orbitrap Exploris 480 mass spectrometer (Thermo Fisher Scientific). Peptide mixtures were loaded onto a 20 cm×0.075 mm Acclaim PepMap 100 C18 trap column (Thermo Fisher Scientific) for desalting and were later separated on a 25 cm×75 μm ID×1.7 μm C18 integrated column (CoAnn Technologies). The peptides were separated with a 150-minute linear gradient from 2% to 32% of solvent B (0.1% formic acid in acetonitrile) at a flow rate of 300 nL/min. An Orbitrap Exploris 480 mass spectrometer (Thermo Fisher Scientific) operated in data dependent mode was used for untargeted HCP detection. For targeted PRM detection, each sample was analyzed under PRM with an isolation window of 2 m/z. In all experiments, a full mass spectrum at 60,000 resolution relative to m/z 200 (normalized AGC target (%) 300, 20 ms maximum injection time, m/z 380-1600) was followed by time-scheduled PRM scans at 15,000 resolution (normalized AGC target (%) 100, 60 ms maximum injection time). HCD was used with 30% NCE.

### Data Analysis

The mass spectrometry raw files were searched against UniProt Cricetulus Griseus (version 2020) with no redundant entries, with Byonic software (version 4.1.10). The mass tolerance was set at 10 ppm, and the fragment mass tolerance was set at 20 ppm. The search criteria included static carbamidomethylation of cysteines (+57.0214 Da) and variable modification of oxidation (+15.9949 Da) of methionine residues. The database search was performed with trypsin digestion with a maximum of two missed cleavages. The HCPs were positively identified when at least two unique peptides were found. PRM data were manually curated within Skyline (version 21.1).

PS80 Degradation Profiling of mAb-3 with 2DLC-CAD

PS80 degradation profiling was performed. mAb-3 containing 0.1% PS80 was diluted to 0.004% in water and injected into a 2D HPLC-CAD system. PS80 was retained on an Oasis Max column (2.1×20 mm, 30 mm), separated by an Acquity BEH C4 column (2.1×50 mm, 1.7 mm) and detected with a Corona Ultra CAD detector.

### Accelerated Hydrolysis of PS80 in Formulated Drug Products

Accelerated hydrolysis of PS80 in mAb-3 to mAb-15 was performed. The internal standard oleic acid-¹³C₁₈ was added into mAbs, to a final concentration of 1 μg/mL, and 10% PS80 stock solution was also added into the mAbs, to a final concentration of 1% PS80. All samples were incubated at 37° C. for 3 or 5 days, and 10 μL of each sample before and after incubation was collected for oleic acid quantification.

Oleic Acid Quantification in mAbs

Oleic acid released from PS80 degradation in the mAb-3 stability sample was quantified by LC-MRM. 90 μL of extraction buffer containing 1 μg/mL oleic acid-¹³C₁₈ in 80% IPA/20% MeOH was added into 10 μL of each mAb-3 stability sample, mixed and incubated at room temperature for 1 hour. The protein was then precipitated by centrifugation at 14,000 rcf at 25° C. for 30 min, and 40 μL of oleic acid containing supernatant was transferred to a 96-well plate for LC-MRM analysis. Oleic acid released through accelerated PS80 hydrolysis in mAb-3 to mAb-15 was quantified with LC-MRM in a similar manner, except that the extraction buffer contained 80% IPA/20% MeOH without the internal standard oleic acid-¹³C₁₈ added into the mAb-3 to mAb-15 samples before and after incubation. Oleic acid and oleic acid-¹³C₁₈ was quantified by monitoring peaks 281.2/281.2 and 299.2/299.2 with an Agilent 6495 QQQ mass spectrometer equipped with an Agilent 1290 Infinity UHPLC (Agilent, Wilmington, Del.). Peak integration was performed in Skyline, and the oleic acid concentration was calculated on the basis of a calibration curve created from a spiked-in oleic acid concentration plot against

$$\frac{peak area of FFA}{peak area of ISTD} .$$

### MY Clipping Measurement Through Intact Mass Analysis of DS-1 to DS-3

Clipping between the amino acid residues methionine and tyrosine (MY clipping) in DS was identified and quantified through intact mass analysis. DS samples were reduced by mixture of 5 μg DS at 0.25 μg/μL with 4 μL of 5×rapid PNGase buffer (New England Biolabs) and incubated at 80° C. for 10 minutes. Deglycosylation was performed by addition of 1 μL of rapid PNGase (New England Biolabs) into the mixture and incubation at 50° C. for 25 minutes. Subsequently, 2 μg of each sample was injected into the LCMS system, separated by reverse phase chromatography with a BioResolve RP mAb Polyphenyl column (2.7 μm, 2.1 mm×50 mm) and detected with a Waters G2S mass spectrometer. MS data were analyzed in MassLynx software from Waters.

### Example 4. Identification of HCPs Through PMLD Untargeted HCP Profiling

ProteoMiner enrichment coupled with limited digestion (PMLD) coupled with targeted PRM analysis was used to quantify HCPs at the sub-ppm level. This enrichment-based targeted quantification further improved the detection to as low as 0.06 ppm, with high accuracy and precision, as compared with other mass spectrometry-based quantification methods. The low detection limit was critical for risk assessment: 0.13 ppm liver carboxylesterase (CES), 2.48 ppm lysosomal acid lipase (LAL), 1.46 ppm palmitoyl-protein thioesterase 1 (PPT-1) and 0.5 ppm cathepsin D were found to have negative effects on drug product stability, whereas 0.5 ppm lipoprotein lipase (LPL), 0.04 ppm CES, 0.8 ppm LAL, 0.49 ppm PPT-1 and 0.3 ppm cathepsin D were found to be safe in allowing drug products to maintain a 2-3 year shelf-life.

The polysorbate (PS) degradation ability of different PS degrading enzymes (PSDEs) is usually evaluated by comparison of activity in spike-in experiments with recombinant proteins. However, the observed lipase activity of recombinant proteins may not represent the activity of endogenous proteins. For example, PS degradation observed from putative phospholipase B-like 2 (PLBD2) can be due to impurities rather than to PLBD2 itself. Whether LPL can degrade PS remains questionable, because neither recombinant CHO LPL nor endogenous LPL below 1.5 ppm shows any lipase activity. ProteoMiner enrichment coupled with limited digestion (PMLD) coupled with targeted PRM analysis provided a quantitative method for the comparison of lipase activity of PSDE through correlation of lipase activity with endogenous PSDE concentrations in DPs. The major cause of PS degradation can be deduced according to the correlation between the PSDE and lipase activity.

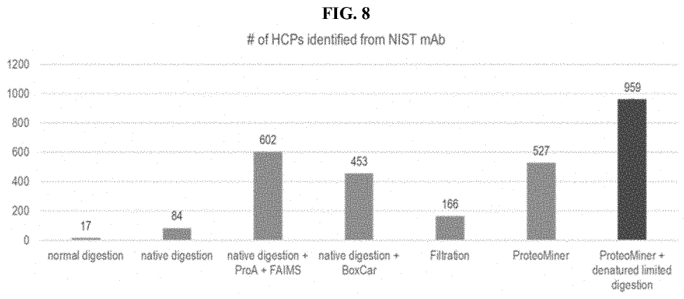

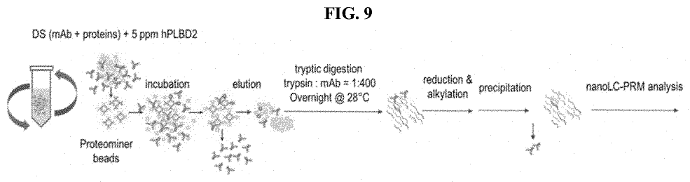

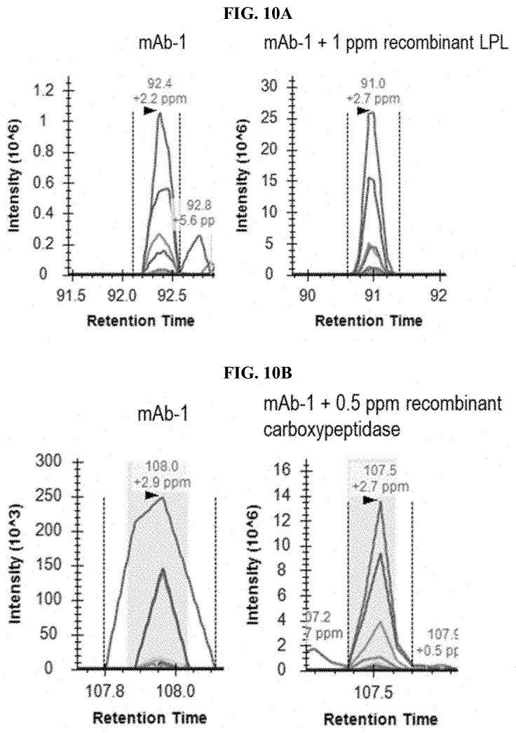

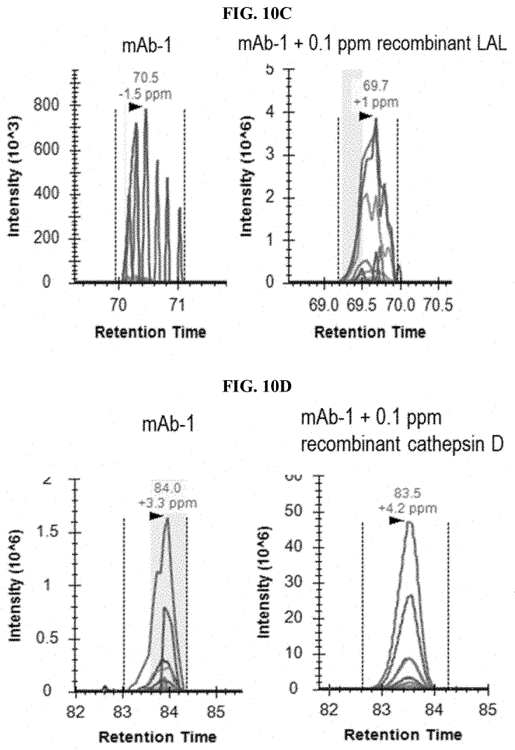

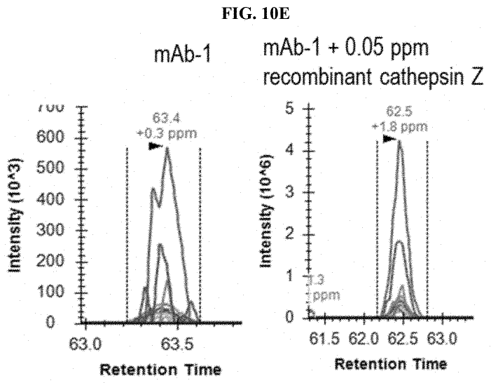

HCP profiling with PMLD was performed for several in-house mAbs to determine the most appropriate matrix for PRM method development. The PMLD workflow is shown in FIG. 9. The detection limit for PMLD was as low as 0.002 ppm. mAb-1 was chosen as the matrix for the standard curve and QC, because it showed minimal interference (≤10% LLOQ level) for the eight spiked-in recombinant CHO protein standards, as shown in FIG. 10. mAb-2 was chosen as the matrix for the standard curves for PPT-1 and LAL, because it was the same mAb as mAb-18 and mAb-19 but did not contain PPT-1 or LAL. mAb-3 contained various levels of HCPs and therefore was used to evaluate intra-run reproducibility. mAb-4 to mAb-16 were antibodies with different levels of lipase activity that were used to access the biologically relevant concentrations of lipase or esterase. The lipase that caused PS80 degradation in mAb-3 to mAb-10 was LAL. The esterase that caused PS80 degradation in mAb-11 to mAb-17 was CES, and the two lipases that contributed to PS80 degradation in mAb-18 and mAb-19 were LAL and PPT-1. DS-1 to DS-3 are fusion proteins used to assess the biologically relevant concentration of cathepsin D.

### Example 5. PMLD-PRM Method Development for HCP Quantification

Table 4 shows the tryptic peptide that was selected for each HCP for targeted PRM quantification. These peptides were chosen because they showed high MS signal intensity, no or low post-translational modification, and no missed cleavages, and were unique to each CHO HCP. hPLBD2 served as the common internal standard for all HCP quantification. The peak area ratio (PAR) of selected HCP peptides was obtained by dividing the peak area of the hPLBD2 peptide by the HCP peptide peak area. The PAR was used to construct a calibration curve for quantification. The relative degree of enrichment of each individual HCP with the PMLD method was comparable to that of hPLBD2 in the same mAb sample, because ProteoMiner is a non-biased enrichment method. In fact, FIG. 11A shows the PRM-based quantification of all eight HCPs in mAb-1, ranging from 0.05 to 5 ppm, followed a linear regression equation with a regression coefficient (R²) above 0.99. This finding suggested that the dynamic range of the PMLD enrichment method followed by targeted PRM analysis was suitable for the quantification of HCPs ranging from sub-ppm to low ppm levels. PMLD-PRM analysis was performed on human PPT-1 and LAL in a range from 0.1 to 20 ppm in mAb-2 to test whether PMLD-PRM analysis could be applied to a wider range of HCPs. FIG. 11B shows that a linear regression line was observed for both human PPT-1 and LAL, with a regression coefficient (R²) above 0.99.

TABLE 4
HCP peptides most frequently identified in shotgun proteomics analysis
for each HCP and hPLBD2

| Host Cell Protein (HCP) | Peptide for PRM Analysis | SEQ ID NO: |
|---|---|---|
| Lipoprotein lipase (LPL) | GLGDVDQLVK | 1 |
| Complement component 1r | SLSNGYLHYITTK | 10 |
| (C1r-A) | | |
| Acid ceramidase (ASAH1) | GQFESYLR | 11 |
| Beta-2-microglobulin | GILLDTSR | 12 |
| Carboxypeptidase | EFSHITFLTIK | 2 |
| Lysosomal acid lipase (LAL) | VNVYTSHSPAGTSVQNLR | 3 |
| Cathepsin D | VSSLPSVTLK | 4 |
| Cathepsin Z | GVNYASITR | 5 |
| Palmitoyl-protein | ETIPLQESTLYTEDR | 13 and 14, |
| thioesterase 1 (PPT-1) | (CHO) | respectively |
| | ETIPLQETSLYTQDR | |
| | (human) | |
| Putative phospholipase | AFIPNGPSPGSR[+10] | 15 |
| B-like 2 (hPLBD2) | | |

The PMLD-PRM method was evaluated for inter-run reproducibility and quantification accuracy with a mixture of five QC standards spiked in mAb-1 with three replicates. The mixture of five QC peptides was prepared with different concentrations of five HCPs: 0.2 ppm cathepsin Z, 0.5 ppm cathepsin D, 1 ppm LAL, 2.5 ppm carboxypeptidase and 5 ppm LPL. The concentration of each QC standard was chosen on the basis of its abundance in mAb-1, to minimize interference in the scans shown in FIG. 10 and to be biologically relevant to drug product quality. As shown in Table 5, the accuracy of detection of the QC peptides of five HCPs ranging from 0.2 ppm to 5 ppm was within 85%-111% of the theoretical values, with variation less than 12% among triplicates. Quantification results are based on peptides of each HCP enriched from mAb-1, and each spiked sample was analyzed in triplicate. The lower limit of quantitation (LLOQ) ranged from 0.06 ppm to 0.66 ppm.

TABLE 5
Accuracy of quantification demonstrated by a spike-in study of five
HCPs

| | Spiked | | | | |
|---|---|---|---|---|---|
| | in | Measured | | | Calculated |
| | Conc. | Conc. | % CV | % | LLOQ |
| Protein Name | (ppm) | (ppm) | (N = 3) | Accuracy | (ppm) |
| cathepsin Z | 0.2 | 0.17 | 11.8% | 15.0% | 0.06 |
| cathepsin D | 0.5 | 0.55 | 10.9% | 10.0% | 0.14 |
| LAL | 1 | 0.91 | 5.0% | 9.2% | 0.14 |
| carboxypeptidase | 2.5 | 2.58 | 8.5% | 3.2% | 0.66 |
| LPL | 5 | 5.36 | 4.5% | 7.2% | 0.43 |

Intra-run reproducibility was evaluated with mAb-3, which contained HCPs at various levels ranging from 0.03 ppm to 4.24 ppm. Three biological replicates of mAb-3 were prepared and analyzed separately on three different days, and six HCPs were evaluated quantitatively, the results of which are shown in Table 6. The precision was within 25% for all HCPs below 0.5 ppm and was within 20% for HCPs at above 0.5 ppm. Quantification results are based on peptides of each HCP enriched from mAb-3, and each sample was analyzed in triplicate.

TABLE 6
Measured concentrations of six HCPs from mAb-3

| | Measured | Measured | Measured | | |
|---|---|---|---|---|---|
| | Conc. | Conc. | Conc. | Avg. | |
| | (ppm) | (ppm) | (ppm) | Measured | CV |
| Protein Name | Run-1 | Run-2 | Run-3 | Conc. (ppm) | % |
| Clr-A | 0.04 | 0.03 | 0.02 | 0.03 | 25.4% |
| cathepsin Z | 0.03 | 0.03 | 0.03 | 0.03 | 0.9% |
| LPL | 0.15 | 0.21 | 0.23 | 0.20 | 19.8% |
| Beta-2- | 0.30 | 0.35 | 0.38 | 0.34 | 11.9% |
| microglobulin | | | | | |
| LAL | 1.20 | 1.54 | 1.10 | 1.28 | 17.8% |
| cathepsin D | 4.56 | 4.56 | 3.60 | 4.24 | 13.1% |

### Example 6. Biologically Relevant Concentrations of Selected HCPs

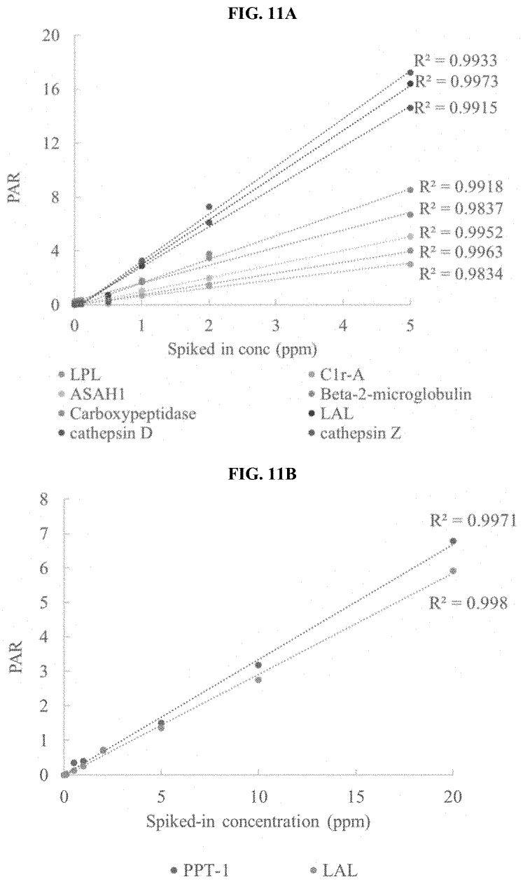

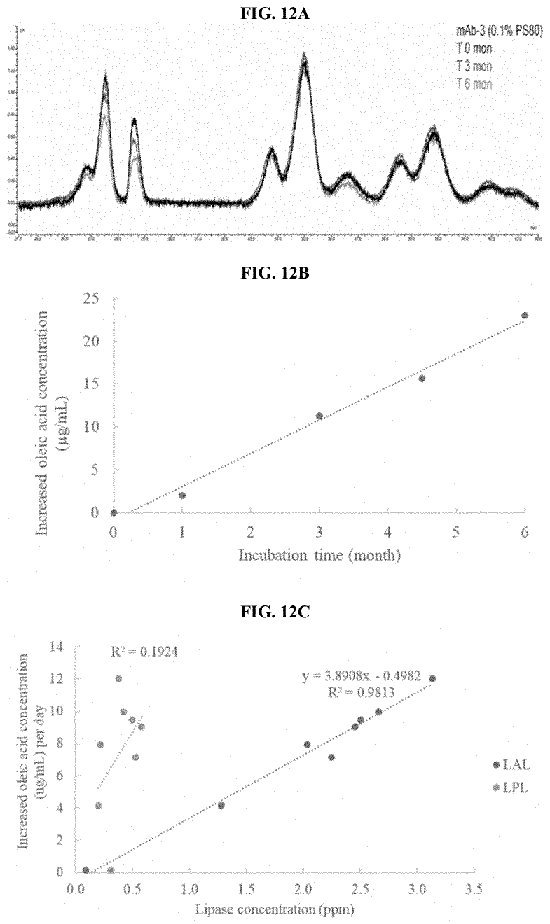

Sub ppm levels of lipases or esterases cause PS degradation in drug products during long-term storage. Table 6 and FIG. 12A show that 1.28 ppm LAL was detected in mAb-3 and led to 20% PS80 degradation, and FIG. 12B shows that 23 μg/mL oleic acid was released at 4-8° C. within 6 months. FIG. 12C shows that LAL, which ranged from 0.1 ppm in mAb-3 to 3.5 ppm in mAb-10, was accurately quantified using PMLD. The regression coefficient (R²) was greater than 0.98 between the oleic acid released from PS80 degradation and the measured LAL concentration. The LAL concentration was determined to be 0.1 ppm in mAb-4 through the PMLD method. FIG. 12C shows that 0.1 ppm LAL in mAb-4 did not cause any visible PS80 degradation within 4 days at 37° C. Therefore, the low detection limit of LAL, of 0.1 ppm, aided in accurate estimation of the potential negative effects of LAL on PS80 degradation and could be used to predict the potential shelf life of drug products. LPL at concentrations below 0.5 ppm was detected in all eight samples from mAb3 to mAb10. FIG. 12C shows that PS80 degradation was not correlated with the concentration of LPL in mAb-3 to mAb-10, with an R² of 0.19, thus suggesting that LPL did not affect PS80. FIG. 12C also demonstrates that 0.5 ppm LAL released 1.45 μg/mL oleic acid from PS80 degradation per day at 37° C.

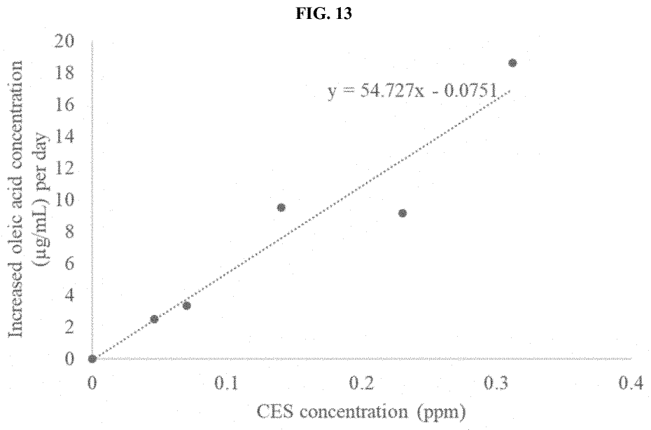

CES is an esterase that, when present at low abundance, can degrade PS80. For example, 20 ppm CES leads to complete depletion of monoesters from PS80 species at 4-8° C. within 24 hours. The concentration of CES in mAb-11 was determined to be 2.3 ppm by quantification through native digestion coupled with MRM. In mAb-12, which is the same mAb as mAb-11 but was obtained through different purification steps, CES was not detected by PMLD, and no PS80 degradation was observed. mAb-13 was formulated by mixture of mAb-11 and mAb-12 at a 1:9 ratio; consequently, the concentration of CES in mAb-13 was determined to be 0.23 ppm. mAb-14 was formulated by mixture of mAb-11 and mAb-12 at a 1:49 ratio, and the concentration of CES in mAb-14 was determined to be 0.046 ppm. mAb-12 to mAb-17 were enriched by PMLD, and the absolute abundance of CES in each sample was calculated as relative abundance with respect to mAb-13 and mAb-14. FIG. 13 shows that a correlation between the increase in oleic acid concentration per day and CES concentration was established. It was estimated that even 0.1 ppm CES would lead to a 5.4 μg/mL oleic acid increase per day under accelerated degradation conditions (37° C.).

PPT-1 and LAL were found to be the possible causes of PS80 degradation in both mAb-18 and mAb-19. In contrast to mAb-3, in which only LAL, but not LPL, was responsible for PS80 degradation, both PPT-1 and LAL played key roles in degrading PS80. Table 4 shows that quantification of CHO PPT-1 was performed with the peptide ETIPLQESTLYTEDR (SEQ ID NO: 13), whereas the calibration curve was created with the human PPT-1 peptide ETIPLQETSLYTQDR (SEQ ID NO: 14), because of the lack of recombinant CHO PPT-1. Given the difference of only a single amino acid residue among the 15 residues, the ionization efficiency was not expected to substantially vary between these two peptides.

Table 7 shows quantification results for PPT-1 and LAL in mAb-18 and mAb-19, as well as measurement of released oleic acid after incubation at 37° C.; 1.8 ppm PPT-1 and 0.39 ppm LAL were detected in mAb-18, thus resulting in an 8.57 μg/mL oleic acid increase per day, respectively, whereas 1.1 ppm PPT-1 and 0.34 ppm LAL were found in mAb-19 and resulted in a 5.28 μg/mL oleic acid increase per day under accelerated degradation conditions (37° C.). On the basis of the mAb-3 results in FIG. 12, 0.5 ppm LAL was found to degrade approximately 1.45 μg/mL oleic acid per day at 37° C. It was estimated that 1 ppm PPT-1 induced an increase of approximately 4 μg/mL oleic acid per day.

TABLE 7
Quantification of PPT-1, LAL and oleic acid increase per day in
mAb-18 and mAb-19

| | | | Oleic acid increase |
|---|---|---|---|
| | | | (pg/mL per day @ |
| | PPT-1 (ppm) | LAL (ppm) | 37° C.) |
| mAb-18 | 1.80 | 0.39 | 8.57 |
| mAb-19 | 1.13 | 0.34 | 5.28 |

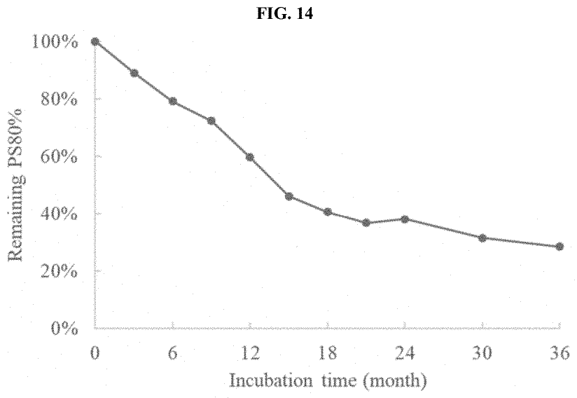

FIG. 14 shows that 71.6% PS80 degradation was observed after mAb-18 was incubated for 36 months at 4-8° C. Sub-visible and visible particles form after 61% degradation of PS80. Although particle formation may vary among different protein formulations, this observation was used to estimate the particle formation time. Using the results from the mAb-18 stability study, it was estimated that approximately 7.3 μg/mL oleic acid released per day under accelerated degradation conditions (37° C.) would result in 61% PS80 degradation with subsequent particle formation in 36 months. Therefore, through a long-term stability study, it was estimated that 1.8 ppm PPT-1, 0.14 ppm CES or 2.5 ppm LAL would be likely to cause particle formation in the DP. The relative lipase activity between different PSDEs was also compared on the basis of the oleic acid increase per day per ppm enzyme, which was calculated to be 2.9, 54.7 and 4.1 μg/mL per day per ppm for LAL, CES and PPT-1, respectively. Therefore, the PS degradation ability of these three enzymes was ranked as CES>PPT-1>LAL.

Lipases and esterases are not the only types of high-risk HCP that must be quantified at sub-ppm levels. FIG. 15 shows that, during elevated stability studies of the drug substances DS-1, DS-2 and DS-3, cathepsin D caused more than 12%, 4% and 0.2% clipping, respectively, between the amino acid residues methionine and tyrosine under 45° C. stress conditions within 6 months. PMLD-PRM quantification analysis was used to determine the cathepsin D concentrations in DS-1, DS-2 and DS-3 were 1.5 ppm, 1.1 ppm and 0.3 ppm cathepsin D, respectively. The results indicated that at concentrations as low as 1.1 ppm or above, cathepsin D cleaves proteins, whereas at 0.3 ppm, it shows negligible protein cleavage.

### Example 7. Using ProteoMiner™ to Enhance the Detection Limit of SV NIST mAbs

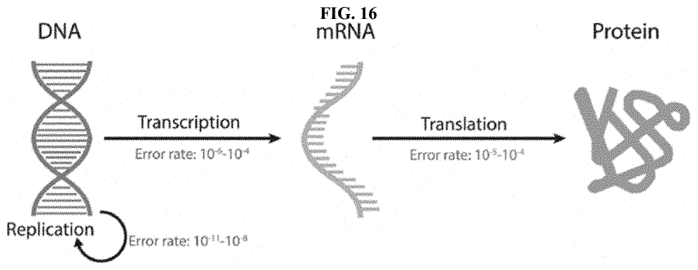

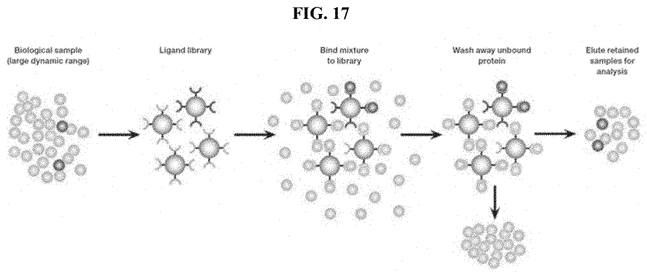

The ProteoMiner™ method for enhanced detection of SV proteins of the present disclosure improves upon the ProteoMiner™ method for HCP identification described by Chen et al. to identify sequence variations within SV NIST mAbs, as shown in FIG. 17. The ProteoMiner™ SV enrichment method includes the steps of contacting a sample comprising at least one more-abundant protein or peptide whose amino acid sequence was not unintentionally altered (e.g., wild-type or recombinant) to a solid support, such as beads, wherein interacting peptide ligands are attached to the solid support and SV NIST mAbs can bind to the interacting peptide ligands, for example ProteoMiner™ beads; washing the solid support using a solution comprising a surfactant to enrich SV NIST mAbs and providing an eluate; subjecting the eluate to denaturation, alkylation and reduction; subjecting the denatured, alkylated and reduced eluate to an enzymatic digestion reaction to generate components of the enriched SV NIST mAbs (e.g., direct digestion); identifying the components of the enriched SV NIST mAbs using a mass spectrometer; and using the identified components to identify amino acid substitutions within the enriched SV NIST mAbs.

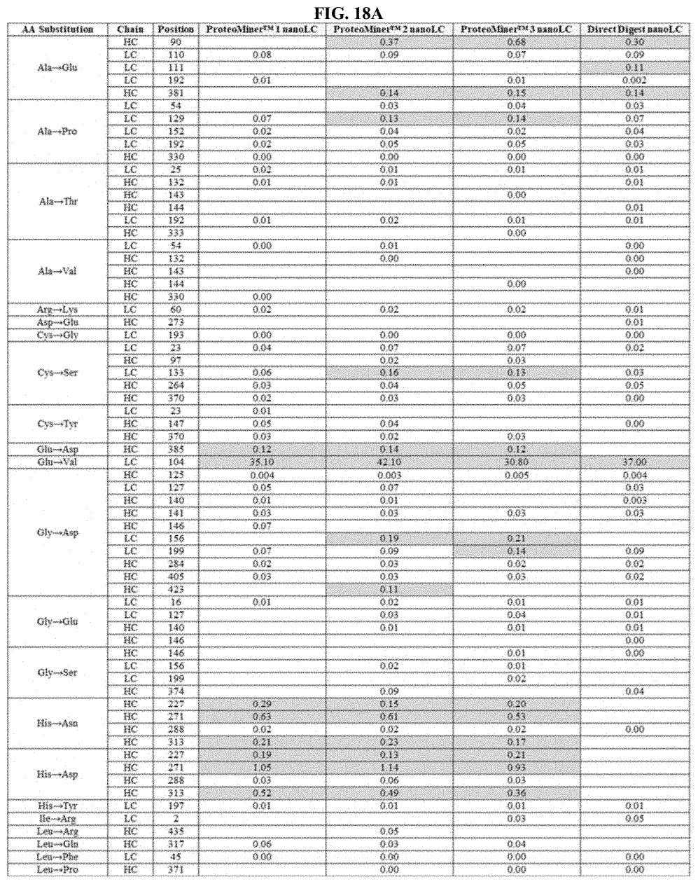

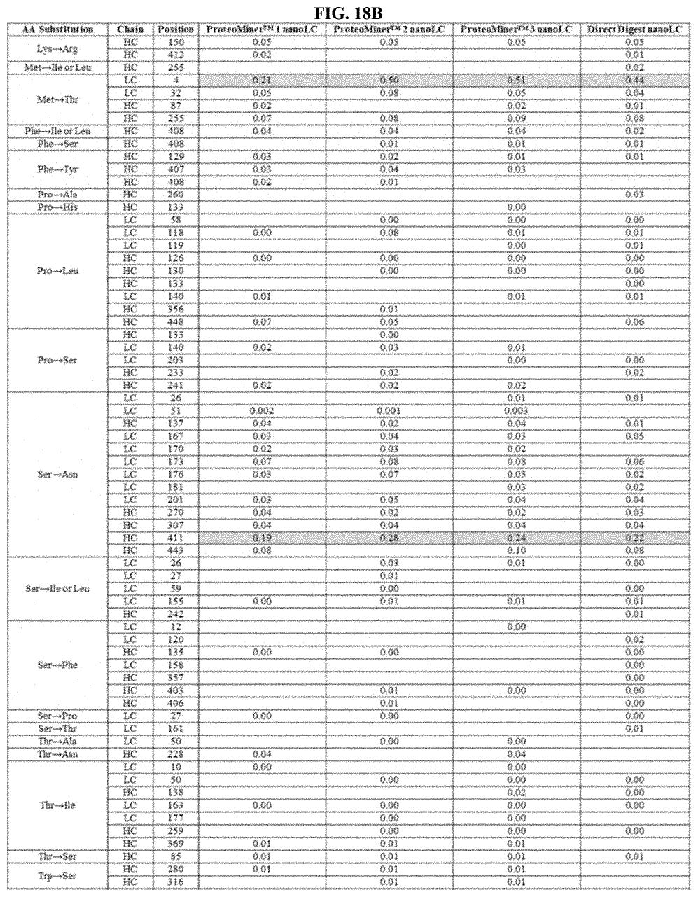

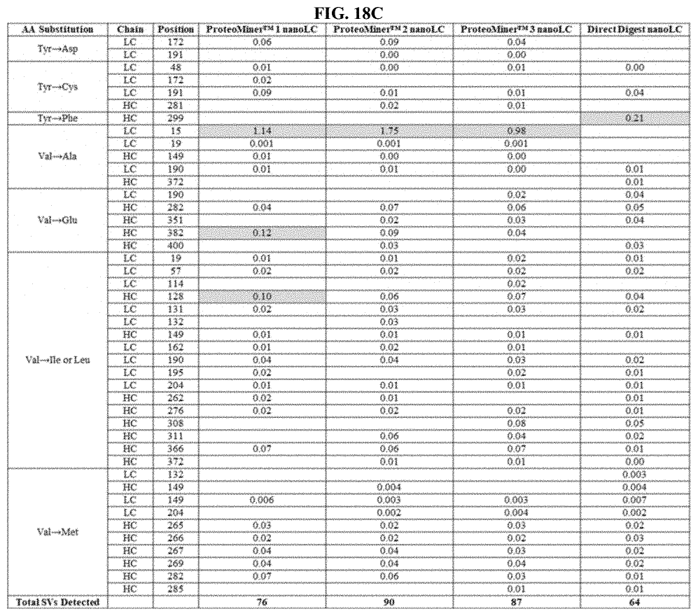

The ProteoMiner™ method for enhanced detection of SV proteins of the present disclosure, nanoLC, or a combination thereof enabled the detection of amino acid substitutions within SV NIST mAbs that included alanine to glutamic acid, proline, threonine or valine, cysteine to glycine, serine or tyrosine, aspartic acid to glutamic acid, glutamic acid to aspartic acid or valine, phenylalanine to serine, tyrosine or leucine or isoleucine, glycine to aspartic acid, glutamic acid or serine, histidine to asparagine, aspartic acid or tyrosine, isoleucine to arginine, lysine to arginine, leucine to arginine, glutamine, phenylalanine or proline, methionine to threonine, leucine or isoleucine, proline to alanine, histidine, leucine or serine, arginine to lysine, serine to asparagine, phenylalanine, proline, threonine, leucine or isoleucine, threonine to alanine, asparagine, isoleucine or serine, valine to alanine, glutamine, methionine, leucine or isoleucine, tryptophan to serine, and tyrosine to aspartic acid, cysteine or phenylalanine, as shown in FIG. 18A, FIG. 18B, and FIG. 18C. The cells highlighted in red show the amino acid substitutions in SV peptides that were enriched using the ProteoMiner™ method for enhanced detection of SV proteins of the present disclosure, nanoLC, or a combination thereof.

### Example 8. Using ProteoMiner™ to Enrich SV NIST mAbs

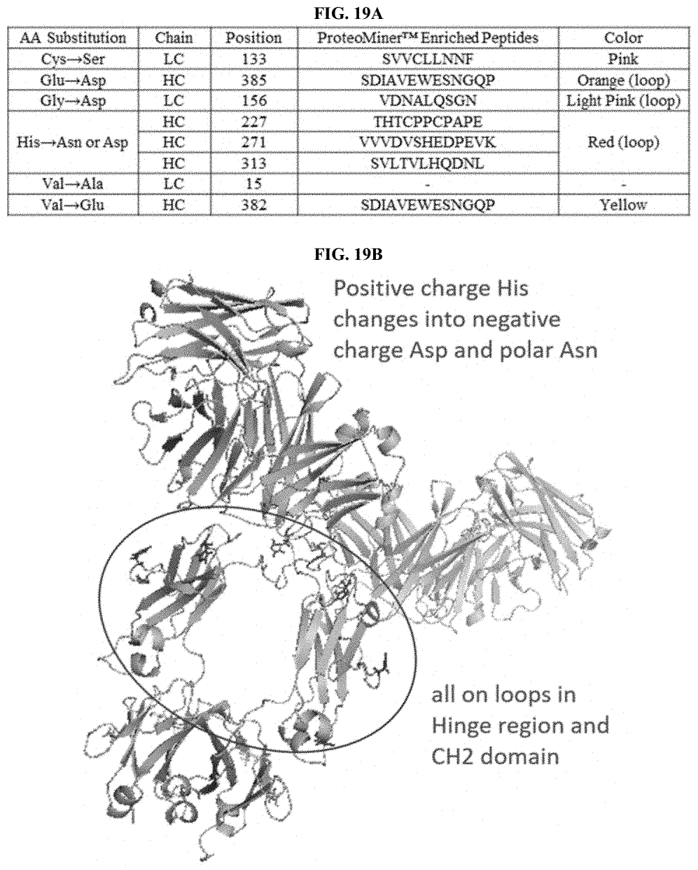

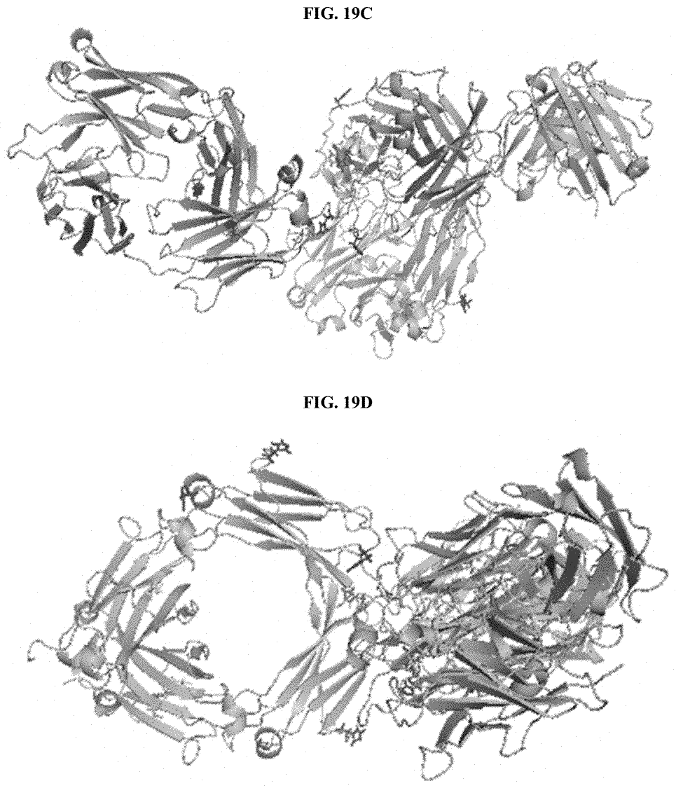

FIG. 19A shows a table of amino acid sequence variations within SV NIST mAbs that were enriched using the ProteoMiner™ method for enhanced detection of SV proteins of the present disclosure, nanoLC, or a combination thereof. The amino acid sequence variations within enriched SV NIST mAbs generally resulted in substitution of an amino acid with a set of physical characteristics for an amino acid with a different set of physical characteristics. Such substitutions effected the three-dimensional protein structure of SV NIST mAbs that were enriched using the ProteoMiner™ method for enhanced detection of SV proteins of the present disclosure, nanoLC, or a combination thereof. FIG. 19B, FIG. 19C, and FIG. 19D show views of the NIST mAb amino acid sequence variations identified and enriched using the ProteoMiner™ SV identification method of the present disclosure within the three-dimensional protein structure of an SV NIST mAb according to an exemplary embodiment.

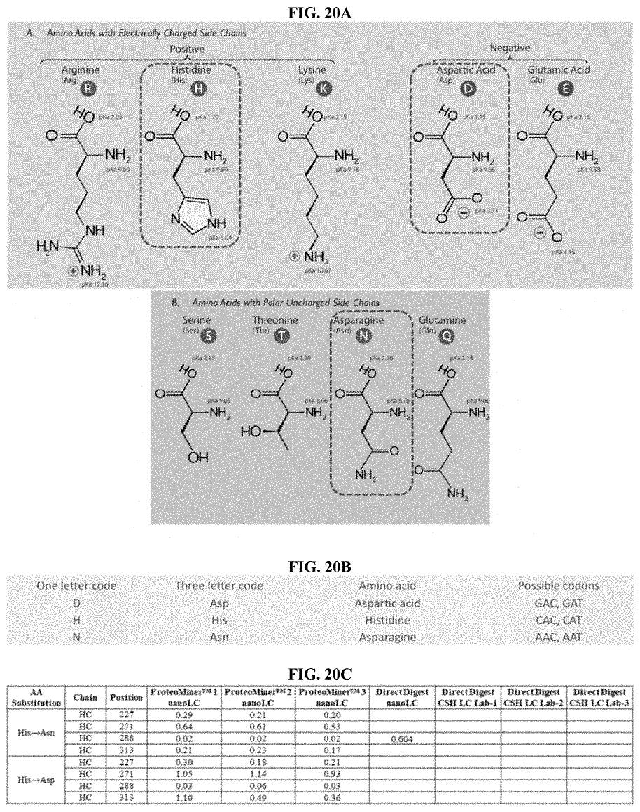

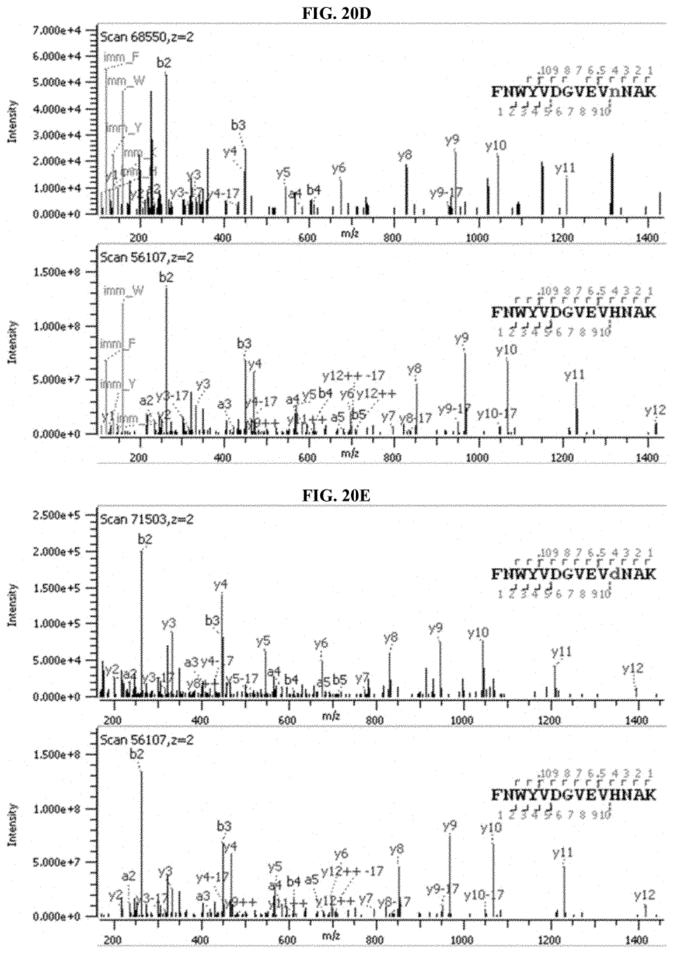

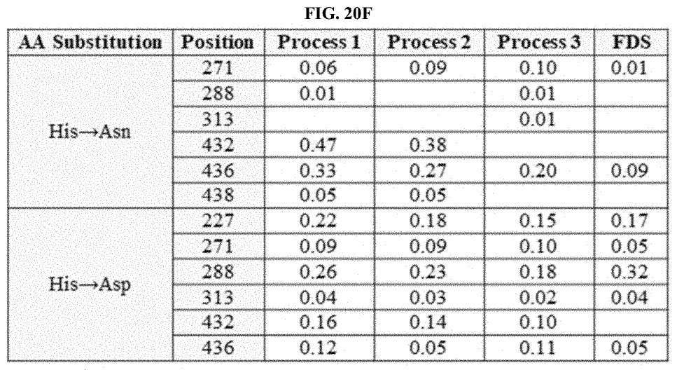

For example, FIG. 20A shows a positively charged histidine that was substituted with a negatively charged aspartic acid or a polar, uncharged asparagine in SV within SV NIST mAbs that were enriched using the ProteoMiner™ method for enhanced detection of SV proteins of the present disclosure, nanoLC, or a combination thereof. FIG. 20B shows possible codon sequences of histidine, aspartic acid, and asparagine, and that a point mutation (e.g., one mutated DNA) can result in substitution of a histidine codon for an aspartic acid or asparagine codon. FIG. 20C shows the NIST mAb histidine to asparagine or aspartic acid sequence variations identified using eluates from NIST mAb direct digests subjected to regular flow CSH LC or nanoLC columns or ProteoMiner™ enriched NIST mAb digests subjected to nanoLC columns according to an exemplary embodiment. FIG. 20C also shows that the ProteoMiner™ method for enhanced detection of SV proteins of the present disclosure enriched SV NIST mAbs with histidine 227, 271, or 313 substitutions, or a combination thereof, for aspartic acid or asparagine.

FIG. 20D shows the MS2 mass spectrum of tryptic peptide product ions detected in an eluate from an NIST mAb direct digest subjected to a regular flow CSH LC column (bottom), and the MS2 mass spectrum of histidine to asparagine SV tryptic peptide product ions detected in an eluate from a ProteoMiner™ enriched NIST mAb digest subjected to a nanoLC column (top), according to an exemplary embodiment. FIG. 20E shows the MS2 mass spectrum of tryptic peptide product ions detected in an eluate from an NIST mAb direct digest subjected to a regular flow CSH LC column (bottom), and the MS2 mass spectrum of histidine to aspartic acid SV tryptic peptide product ions detected in an eluate from a ProteoMiner™ enriched NIST mAb digest subjected to a nanoLC column (top), according to an exemplary embodiment. Similarly, the ProteoMiner™ method for enhanced detection of SV proteins of the present disclosure could enrich CHO IgG1 mAbs with histidine to asparagine or aspartic acid sequence variations. FIG. 20F shows that the ProteoMiner™ method for enhanced detection of SV proteins of the present disclosure enriched CHO IgG1 mAbs with histidine 432 or 436 substitutions, or a combination thereof, for asparagine. FIG. 20F also shows that the ProteoMiner™ method for enhanced detection of SV proteins of the present disclosure enriched CHO IgG1 mAbs with histidine 227, 271, 288, 432, or 436 substitutions, or a combination thereof, for aspartic acid.

### Example 9. ProteoMiner™ Enriches SV NIST mAbs that have an Altered Three-Dimensional Protein Structure

The amino acid sequence variations within SV NIST mAbs that were not enriched using the ProteoMiner™ method for enhanced detection of SV proteins of the present disclosure generally resulted in substitution of an amino acid with a set of physical characteristics for an amino acid with the same set of physical characteristics. Such substitutions did not affect the three-dimensional protein structure of SV NIST mAbs that were not enriched using the ProteoMiner™ method for enhanced detection of SV proteins of the present disclosure, nanoLC, or a combination thereof.

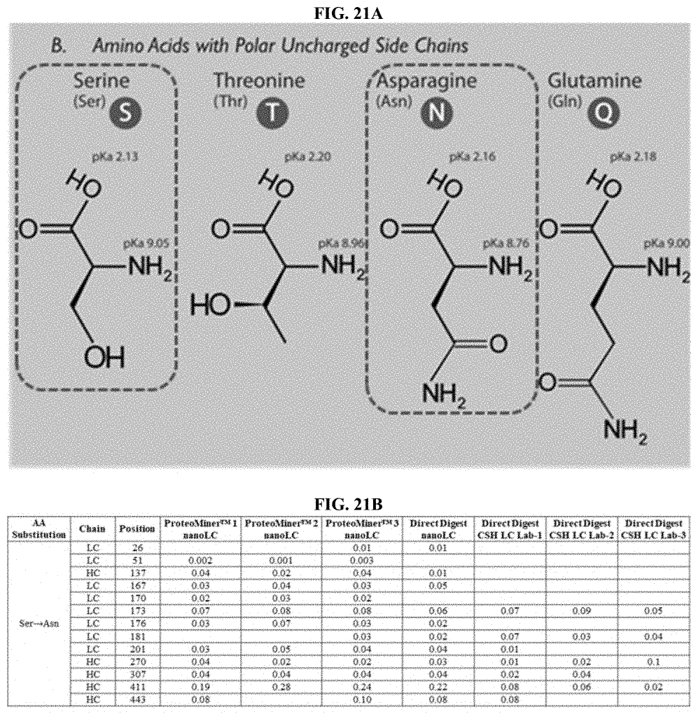

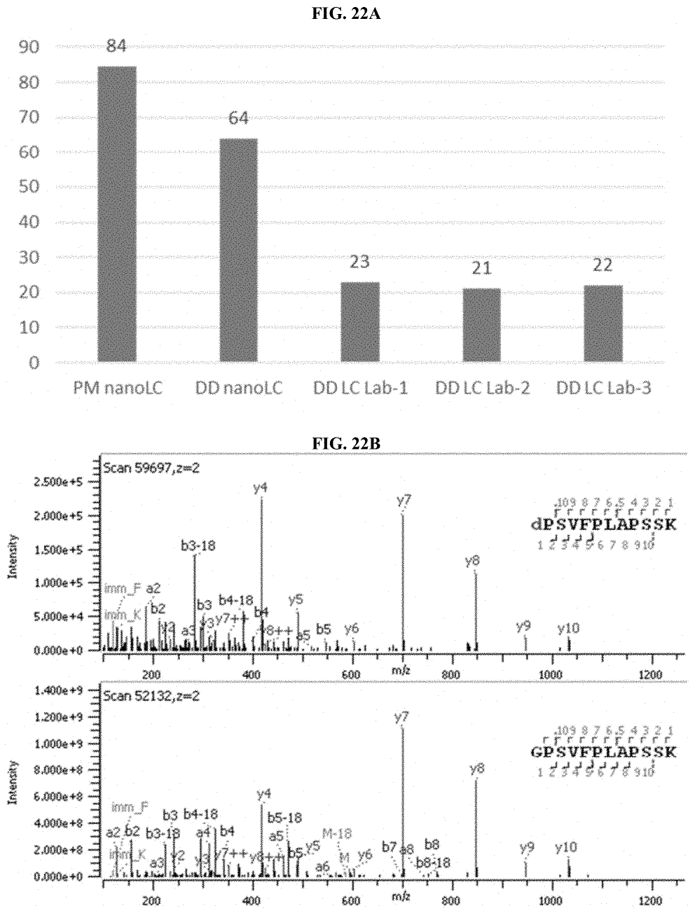

For example, FIG. 21A shows a polar, uncharged serine that was substituted with a polar, uncharged asparagine in SV within SV NIST mAbs that were not enriched using the ProteoMiner™ method for enhanced detection of SV proteins of the present disclosure, nanoLC, or a combination thereof. FIG. 21B shows the NIST mAb serine to asparagine sequence variations identified using eluates from NIST mAb direct digests subjected to regular flow CSH LC or nanoLC columns or ProteoMiner™ eluate NIST mAb digests subjected to nanoLC columns according to an exemplary embodiment.

### Example 10. ProteoMiner™ Increases the Number of SVs Detected Compared to Direct Digestion and Regular Flow LC or nanoLC

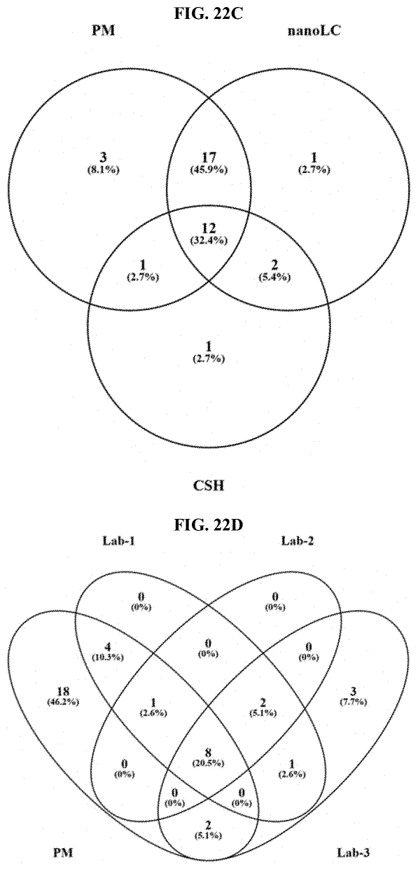

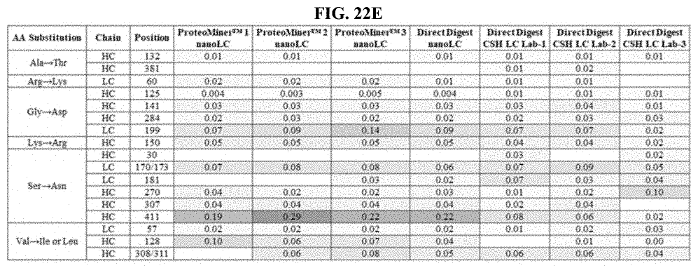

The number of NIST mAb amino acid sequence variations identified (SVA >0.01%) using eluates from NIST mAb direct digests subjected to regular flow CSH LC or nanoLC columns was fewer than ProteoMiner™ eluate NIST mAb digests subjected to nanoLC columns, as shown in FIG. 22A. While the ProteoMiner™ method for enhanced detection of SV proteins of the present disclosure can enrich certain SV proteins, the method generally enhances detection of SV proteins even without enrichment. Additionally, nanoLC improved the sensitivity for detecting SV proteins. FIG. 22B shows the MS2 mass spectrum of tryptic peptide product ions detected in an eluate from an NIST mAb direct digest subjected to a regular flow CSH LC column (bottom), and the MS2 mass spectrum of glycine to aspartic acid SV tryptic peptide product ions detected (SVA as low as 0.004%) in an eluate from a ProteoMiner™ enriched NIST mAb digest subjected to a nanoLC column (top), according to an exemplary embodiment.

Serine, glycine, and valine SV NIST mAbs were used to further evaluate the ProteoMiner™ method for enhanced detection of SV proteins of the present disclosure. FIG. 22C shows the number of NIST mAb serine, glycine, and valine sequence variations identified using eluates from NIST mAb direct digests subjected to regular flow CSH LC or nanoLC columns or ProteoMiner™ enriched NIST mAb digests subjected to nanoLC columns according to an exemplary embodiment. FIG. 22C shows that about 50% more SVs could be detected using direct digests subjected to r nanoLC columns or ProteoMiner™ eluate NIST mAb digests subjected to nanoLC columns than direct digests subjected to regular flow CSH LC columns. FIG. 22D shows the number of NIST mAb serine, glycine, or valine sequence variations identified by three labs using eluates from NIST mAb direct digests subjected to regular flow CSH LC columns or ProteoMiner™ enriched NIST mAb digests subjected to nanoLC columns according to an exemplary embodiment. FIG. 22D shows that about 85% of SVs were detected using the ProteoMiner™ method for enhanced detection of SV proteins of the present disclosure, and that about 92.3% of SVs were detected using the ProteoMiner™ method for enhanced detection of SV proteins of the present disclosure, nanoLC, or a combination thereof.

FIG. 22E shows the NIST mAb alanine to threonine, glycine to aspartic acid, serine to asparagine, valine to leucine or isoleucine, arginine to lysine, and lysine to arginine sequence variations identified by three labs using eluates from NIST mAb direct digests subjected to regular flow CSH LC columns or NIST mAb direct digests or ProteoMiner™ enriched NIST mAb digests subjected to nanoLC columns according to an exemplary embodiment. The ProteoMiner™ method for enhanced detection of SV proteins of the present disclosure or nanoLC detected 15 of the 17 SV NIST mAbs detected by subjecting NIST mAb direct digests to regular flow CSH LC columns. Additionally, FIG. 22E shows that 1 of the 17 SV NIST mAbs detected by subjecting NIST mAb direct digests to regular flow CSH LC columns showed a higher SV percentage using nanoLC, whereas others showed a similar relative abundance.

### Example 11. NanoLC can Cause an Overestimation of SVA

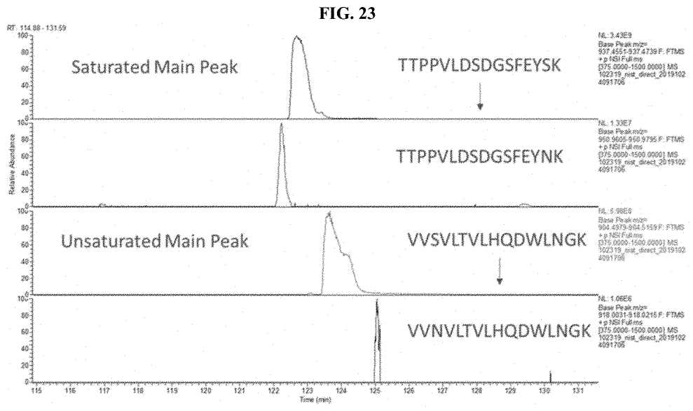

FIG. 23 shows unsaturated (bottom) and saturated (top) peaks in the MS2 mass spectrum of tryptic peptide product ions (e.g., VVSVLTVLHQDWLNGK (SEQ ID NO: 6) and TTPPVLDSDGSFEYSK (SEQ ID NO: 7)) and serine to asparagine SV tryptic peptide product ions (e.g., VVNVLTVLHQDWLNGK (SEQ ID NO: 8) and TTPPVLDSDGSFEYNK (SEQ ID NO: 9)) detected in an eluate from a digested ProteoMiner™ NIST mAb eluate subjected to a nanoLC column according to an exemplary embodiment. Saturated peaks in the MS2 mass spectrum of SV and non-SV peptide product ions can obscure the relative abundance of SV and non-SV proteins, leading to an overestimation in the relative abundance of an SVA.

### Example 12. NanoLC can Improve MS2 Spectra

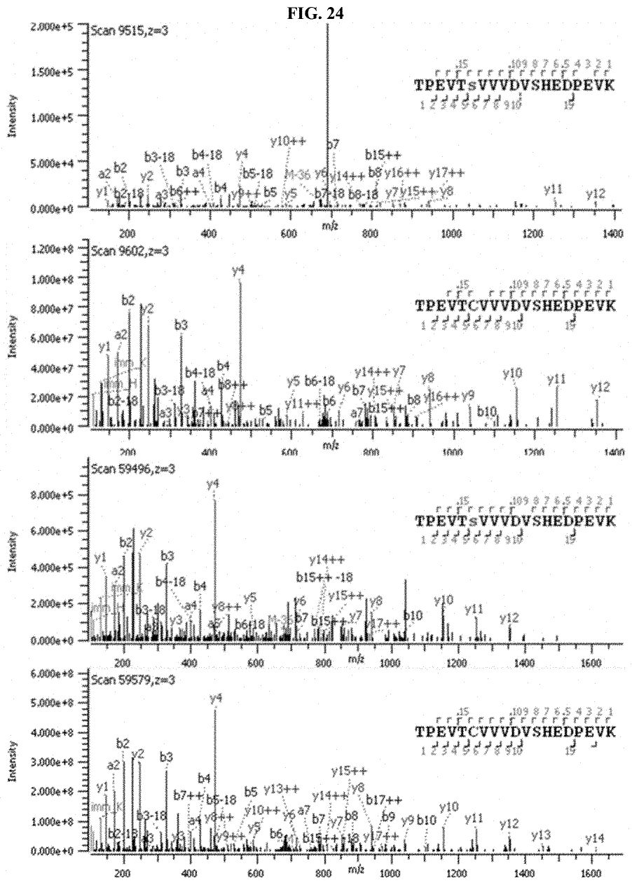

NanoLC can improve MS2 spectra by increasing the signal. For example, FIG. 24 shows analyzing an eluate from an NIST mAb direct digest subjected to a regular flow CSH LC column using a mass spectrometer produces larger peaks in the MS2 mass spectrum of tryptic peptide product ions (Scan 9602, z=3) than in the MS2 mass spectrum of cysteine to serine SV tryptic peptide product ions (Scan 9515, z=3). Conversely, analyzing an eluate from a digested ProteoMiner™ NIST mAb eluate subjected to a nanoLC column using a mass spectrometer produces smaller peaks in the MS2 mass spectrum of tryptic peptide product ions (Scan 59496, z=3) than in the MS2 mass spectrum of cysteine to serine SV tryptic peptide product ions (Scan 59579, z=3) according to an exemplary embodiment.

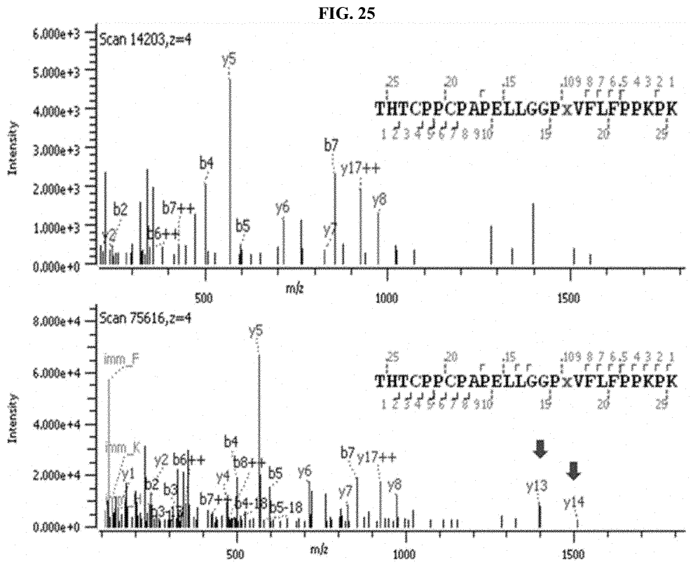

NanoLC can also improve MS2 spectra by enabling generation of y-ions. For example, FIG. 25 shows a mass spectrometer does not generate y-ions in the MS2 mass spectrum of serine to leucine or isoleucine SV tryptic peptide product ions using an eluate from an NIST mAb direct digest subjected to a regular flow CSH LC column (Scan 14203, z=4). Conversely, a mass spectrometer generates y-ions in the MS2 mass spectrum of serine to leucine or isoleucine SV tryptic peptide product ions using an eluate from a digested ProteoMiner™ NIST mAb eluate subjected to a nanoLC column (Scan 75616, z=4) according to an exemplary embodiment.

What is claimed is:

1. A method of identifying host cell protein (HCP) impurities in a sample, comprising:
(a) contacting a sample including at least one high-abundance peptide or protein and at least one HCP impurity to a solid support, wherein said solid support is attached to interacting peptide ligands capable of interacting with said at least one HCP impurity;
(b) washing said solid support to provide an eluate comprising at least one enriched HCP impurity;
(c) subjecting said eluate to an enzymatic digestion condition to generate at least one component of said at least one enriched HCP impurity, wherein said enzymatic digestion condition does not fully digest all proteins in said eluate;
(d) identifying said at least one component of said at least one enriched HCP impurity using a mass spectrometer; and
(e) using the identification of said at least one component to identify said at least one enriched HCP impurity.

2. The method of claim 1, wherein said solid support is washed using a surfactant, wherein said surfactant is a phase transfer surfactant, an ionic surfactant, an anionic surfactant, a cationic surfactant, or combinations thereof.

3. The method of claim 2, wherein said surfactant is sodium deoxycholate, sodium lauryl sulfate, sodium dodecylbenzene sulphonate, or combinations thereof.

4. The method of claim 1, wherein a concentration of said surfactant is about 12 mM.

5. The method of claim 3, wherein said surfactant comprises about 12 mM sodium deoxycholate and about 12 mM sodium lauryl sulfate.

6. The method of claim 1, wherein a concentration of said at least one high-abundance peptide or protein is at least about 1000 times, about 10,000 times, about 100,000 times or about 1,000,000 times higher than a concentration of said at least one HCP impurity.

7. The method of claim 1, wherein said interacting peptide ligands are a library of combinatorial hexapeptide ligands.

8. The method of claim 1, wherein said at least one high-abundance peptide or protein is an antibody, a bispecific antibody, an antibody fragment, a Fab region of an antibody, an antibody-drug conjugate, a fusion protein, a recombinant protein, a protein pharmaceutical product, or a drug.

9. The method of claim 1, wherein an enzyme of said enzymatic digestion condition is trypsin.

10. The method of claim 9, wherein said enzymatic digestion condition includes trypsin at an enzyme to substrate ratio of less than about 1:200.

11. The method of claim 10, wherein said enzymatic digestion condition includes trypsin at an enzyme to substrate ratio of about 1:400, about 1:1000, about 1:2500, or about 1:10000.

12. The method of claim 1, wherein said at least one enriched HCP impurity is not subjected to denaturation prior to being subjected to said enzymatic digestion condition.

13. The method of claim 1, wherein said mass spectrometer is an electrospray ionization mass spectrometer, nano-electrospray ionization mass spectrometer, or a triple quadrupole mass spectrometer, wherein the mass spectrometer is coupled to a liquid chromatography system.

14. The method of claim 1, wherein said mass spectrometer is capable of performing LC-MS (liquid chromatography-mass spectrometry) or a LC-MRM-MS (liquid chromatography-multiple reaction monitoring-mass spectrometry) analyses.

15. The method of any one of claims 1-14, further comprising quantifying said at least one enriched HCP impurity using said mass spectrometer, wherein a detection limit of said at least one enriched HCP impurity is about 0.003-0.006 ppm.

16. A method of identifying sequence variant (SV) peptides or proteins in a sample, wherein at least one amino acid of a SV peptide or protein unintentionally differs from a wild-type peptide or protein, comprising:
(a) contacting a sample containing at least one wild-type peptide or protein and at least one SV peptide or protein to a solid support, wherein said solid support is attached to interacting peptide ligands capable of interacting with said at least one SV peptide or protein;
(b) washing said solid support to provide a first eluate comprising at least one enriched SV peptide or protein;
(c) subjecting said first eluate to an enzymatic digestion condition to generate at least one component of said at least one enriched SV peptide or protein;
(d) subjecting said first eluate with the at least one component of the at least one enriched SV peptide or protein to a liquid chromatography system to produce a second eluate;
(e) subjecting said second eluate to mass spectrometry;
(f) identifying said at least one component of said at least one enriched SV peptide or protein using a mass spectrometer; and
(g) using the identification of said at least one component to identify said at least one enriched SV peptide or protein in said sample.

17. The method of claim 16, wherein said enzymatic digestion condition is a direct digestion.

18. The method of claim 17, wherein said liquid chromatography system comprises a nanoscale liquid chromatography (nanoLC) column or a regular flow charged surface hybrid (CSH) column.

19. The method of claim 18, wherein said enzymatic digestion condition does not fully digest all proteins in said first eluate.

20. The method of claim 19, wherein said solid support is washed using a surfactant, wherein said surfactant is a phase transfer surfactant, an ionic surfactant, an anionic surfactant, a cationic surfactant, or combinations thereof.

21. The method of claim 20, wherein said surfactant is sodium deoxycholate, sodium lauryl sulfate, sodium dodecylbenzene sulphonate, or combinations thereof.

22. The method of claim 19, wherein a concentration of said surfactant is about 12 mM.

23. The method of claim 20, wherein said surfactant comprises about 12 mM sodium deoxycholate and about 12 mM sodium lauryl sulfate.

24. The method of claim 19, wherein a concentration of said at least one more-abundant wild-type peptide or protein is at least about 1000 times, about 10,000 times, about 100,000 times or about 1,000,000 times higher than a concentration of said at least one SV peptide or protein.

25. The method of claim 19, wherein said interacting peptide ligands are a library of combinatorial hexapeptide ligands.

26. The method of claim 19, wherein said at least one more-abundant wild-type peptide or protein and said at least one SV peptide or protein are an antibody, a bispecific antibody, an antibody fragment, a Fab region of an antibody, an antibody-drug conjugate, a fusion protein, a recombinant protein, a protein pharmaceutical product, or a drug.

27. The method of claim 19, wherein an enzyme of said enzymatic digestion condition is trypsin.

28. The method of claim 27, wherein said enzymatic digestion condition includes trypsin at an enzyme to substrate ratio of less than about 1:200.

29. The method of claim 28, wherein said enzymatic digestion condition includes trypsin at an enzyme to substrate ratio of about 1:400, about 1:1000, about 1:2500, or about 1:10000.

30. The method of claim 19, wherein said at least one enriched SV peptide or protein is not subjected to denaturation prior to being subjected to said enzymatic digestion condition.

31. The method of claim 19, wherein said mass spectrometer is an electrospray ionization mass spectrometer, nano-electrospray ionization mass spectrometer, or a triple quadrupole mass spectrometer, wherein the mass spectrometer is coupled to said liquid chromatography system.

32. The method of claim 19, wherein said mass spectrometer is capable of performing LC-MS (liquid chromatography-mass spectrometry) or a LC-MRM-MS (liquid chromatography-multiple reaction monitoring-mass spectrometry) analyses.

33. The method of any one of claims 19-32, further comprising quantifying said at least one enriched SV peptide or protein using said mass spectrometer, wherein a detection limit of said at least one enriched SV peptide or protein is about 0.003-0.006 ppm.

34. A method of identifying host cell protein (HCP) impurities in a sample, comprising:
(a) contacting a sample including at least one high-abundance peptide or protein and at least one HCP impurity to a solid support, wherein said solid support is attached to interacting peptide ligands capable of interacting with said at least one HCP impurity;
(b) washing said solid support to provide an eluate comprising at least one enriched HCP impurity;
(c) subjecting said eluate to an enzymatic digestion condition to generate at least one component of said at least one enriched HCP impurity, wherein said enzymatic digestion condition does not fully digest all proteins in said eluate;
(d) identifying said at least one component of said at least one enriched HCP impurity using parallel reaction monitoring-mass spectrometry; and
(e) using the identification of said at least one component to identify said at least one enriched HCP impurity.

35. The method of claim 34, wherein said solid support is washed using a surfactant, wherein said surfactant is a phase transfer surfactant, an ionic surfactant, an anionic surfactant, a cationic surfactant, or combinations thereof.

36. The method of claim 35, wherein said surfactant is sodium deoxycholate, sodium lauryl sulfate, sodium dodecylbenzene sulphonate, or combinations thereof.

37. The method of claim 34, wherein a concentration of said surfactant is about 12 mM.

38. The method of claim 36, wherein said surfactant comprises about 12 mM sodium deoxycholate and about 12 mM sodium lauryl sulfate.

39. The method of claim 34, wherein a concentration of said at least one high-abundance peptide or protein is at least about 1000 times, about 10,000 times, about 100,000 times, about 1,000,000 times, about 10,000,000 times, about 100,000,000 times or about 1,000,000,000 times higher than a concentration of said at least one HCP impurity.

40. The method of claim 34, wherein said interacting peptide ligands are a library of combinatorial hexapeptide ligands.

41. The method of claim 34, wherein said at least one high-abundance peptide or protein is an antibody, a bispecific antibody, an antibody fragment, a Fab region of an antibody, an antibody-drug conjugate, a fusion protein, a recombinant protein, a protein pharmaceutical product, or a drug.

42. The method of claim 34, wherein an enzyme of said enzymatic digestion condition is trypsin.

43. The method of claim 42, wherein said enzymatic digestion condition includes trypsin at an enzyme to substrate ratio of less than about 1:200.

44. The method of claim 43, wherein said enzymatic digestion condition includes trypsin at an enzyme to substrate ratio of about 1:400, about 1:1000, about 1:2500, or about 1:10000.

45. The method of claim 34, wherein said at least one enriched HCP impurity is not subjected to denaturation prior to being subjected to said enzymatic digestion condition.

46. The method of claim 34, wherein said mass spectrometer is an electrospray ionization mass spectrometer, nano-electrospray ionization mass spectrometer, or a triple quadrupole mass spectrometer, wherein the mass spectrometer is coupled to a liquid chromatography system.

47. The method of claim 34, wherein the sample includes an internal standard.

48. The method of claim 47, wherein said internal standard is labeled with a heavy isotope.

49. The method of claim 48, wherein said internal standard is hPLBD2.


SEQUENCE LISTING (29 sequences)

SEQ ID NO: 1 - protein, 10 aa, synthetic construct
GLGDVDQLVK

SEQ ID NO: 2 - protein, 11 aa, synthetic construct
EFSHITFLTIK

SEQ ID NO: 3 - protein, 18 aa, synthetic construct
VNVYTSHSPAGTSVQNLR

SEQ ID NO: 4 - protein, 10 aa, synthetic construct
VSSLPSVTLK

SEQ ID NO: 5 - protein, 9 aa, synthetic construct
GVNYASITR

SEQ ID NO: 6 - protein, 16 aa, synthetic construct
VVSVLTVLHQDWLNGK

SEQ ID NO: 7 - protein, 16 aa, synthetic construct
TTPPVLDSDGSFEYSK

SEQ ID NO: 8 - protein, 16 aa, synthetic construct
VVNVLTVLHQDWLNGK

SEQ ID NO: 9 - protein, 16 aa, synthetic construct
TTPPVLDSDGSFEYNK

SEQ ID NO: 10 - protein, 13 aa, synthetic construct
SLSNGYLHYITTK

SEQ ID NO: 11 - protein, 8 aa, synthetic construct
GQFESYLR

SEQ ID NO: 12 - protein, 8 aa, synthetic construct
GILLDTSR

SEQ ID NO: 13 - protein, 15 aa, unidentified
ETIPLQESTLYTEDR

SEQ ID NO: 14 - protein, 15 aa, Homo sapiens
ETIPLQETSLYTQDR

SEQ ID NO: 15 - protein, 12 aa, Homo sapiens
AFIPNGPSPGSR

SEQ ID NO: 16 - protein, 9 aa, synthetic construct
SVVCLLNNF

SEQ ID NO: 17 - protein, 13 aa, synthetic construct
SDIAVEWESNGQP

SEQ ID NO: 18 - protein, 9 aa, synthetic construct
VDNALQSGN

SEQ ID NO: 19 - protein, 11 aa, synthetic construct
THTCPPCPAPE

SEQ ID NO: 20 - protein, 13 aa, synthetic construct
VVVDVSHEDPEVK

SEQ ID NO: 21 - protein, 11 aa, synthetic construct
SVLTVLHQDNL

SEQ ID NO: 22 - protein, 14 aa, synthetic construct
FNWYVDGVEVNNAK

SEQ ID NO: 23 - protein, 14 aa, synthetic construct
FNWYVDGVEVHNAK

SEQ ID NO: 24 - protein, 14 aa, synthetic construct
FNWYVDGVEVDNAK

SEQ ID NO: 25 - protein, 12 aa, synthetic construct
DPSVFPLAPSSK

SEQ ID NO: 26 - protein, 12 aa, synthetic construct
GPSVFPLAPSSK

SEQ ID NO: 27 - protein, 19 aa, synthetic construct
TPEVTSVVVDVSHEDPEVK

SEQ ID NO: 28 - protein, 19 aa, synthetic construct
TPEVTCVVVDVSHEDPEVK

SEQ ID NO: 29 - protein, 26 aa, synthetic construct
  VARIANT 17: note = Any amino acid
THTCPPCPAPELLGGPXVFLFPPKPK